(19) United States
(12) Patent Application Publication
(10) Pub. No.: US 2011/0293714 A1
(43) Pub. Date: Dec. 1, 2011

(54) PHARMACEUTICAL COMPOSITIONS SUITABLE FOR ORAL ADMINISTRATION OF DERIVATIZED INSULIN PEPTIDES

(75) Inventor: Florian Anders Föger, Frederiksberg C (DK)

(73) Assignee: NOVO NORDISK A/S, BGSVAERD (DK)

(21) Appl. No.: 13/131,608

(22) PCT Filed: Sep. 18, 2009
(86) PCT No.: PCT/EP09/62126

§ 371 Date: Aug. 11, 2011

(30) Foreign Application Priority Data
Nov. 28, 2008 (EP) 08170231.8
Mar. 13, 2009 (EP) PCT/EP2009/053017

(51) Int. Cl. A61K 38/28; A61P 3/10; A61K 9/48

(52) U.S. Cl. 424/455; 514/5.9

(57) ABSTRACT

The invention is related to a water-free liquid or semisolid pharmaceutical composition comprising a derivatized insulin peptide, at least one polar organic solvent and at least one lipophilic component and a method of treatment using such.

### FIELD OF THE INVENTION

The invention is related to a water-free liquid or semisolid pharmaceutical composition comprising a derivatized insulin peptide, at least one polar organic solvent and at least one lipophilic component and a method of treatment using such.

### BACKGROUND OF THE INVENTION

Diabetes mellitus is a metabolic disorder in which the ability to utilize glucose is partly or completely lost which may be treated with e.g. insulin.

The general approach for insulin delivery is parenteral administration which is invasive and inconvenient. Therefore non-invasive routes like oral delivery of protein based pharmaceuticals are increasingly investigated. Administration of therapeutic peptides or proteins such as insulin peptides is however often limited to parenteral routes rather than the preferred oral administration due to several barriers such as enzymatic degradation in the gastrointestinal (GI) tract, drug efflux pumps, insufficient and variable absorption from the intestinal mucosa, as well as first pass metabolism in the liver. Human insulin is degraded by various digestive enzymes found in the stomach (pepsin), in the intestinal lumen (chymotrypsin, trypsin, elastase, carboxypeptidases, etc.) and in the mucosal surfaces of the GI tract (aminopeptidases, carboxypeptidases, enteropeptidases, dipeptidyl peptidases, endopeptidases, etc.).

This is unfortunate because many peptides and many proteins have been proven to be clinically effective and could have more widespread use if easy to administer and acceptable to recipients.

Recent formulation designs for oral protein/peptide delivery include co-formulations with protease inhibitors, permeation enhancers, polymer-based delivery systems and insulin conjugates.

A useful vehicle for oral administration of a drug to a mammal, e.g., a human, is in the form of a microemulsion preconcentrate, also called SMEDDS (self microemulsifying drug delivery systems, or SEDDS (self emulsifying drug delivery systems). SEDDS or SMEDDS, e.g., includes at least one oil or other lipophilic ingredients, at least one surfactant, optional hydrophilic ingredients, and any other agents or excipients as needed. When the components of the system contact an aqueous medium, e.g., water, a microemulsion or emulsion spontaneously forms, such as an oil-in-water emulsion or microemulsion, with little or no agitation. Microemulsions are thermodynamically stable system comprising two immiscible liquids, in which one liquid is finely divided into the other because of the presence of a surfactant(s). The microemulsion formed, appears to be e.g., clear or translucent, slightly opaque, opalescent, non-opaque or substantially non-opaque because of the low particle size of the dispersed phase.

WO 2006/035418, related to pharmaceutical formulations comprising a plurality of seamless minicapsules, discloses an insulin SEDDS composition comprising a modified vegetable oil, a surfactant, a co-solvent, a bile salt, insulin and leupeptin.

There is however still a need for physically and chemically stable pharmaceutical compositions comprising a derivatized insulin for oral administration. The present invention thus provides particularly suitable compositions for oral administration containing derivatized insulin having particularly interesting bioavailability characteristics, particularly interesting pharmacokinetic characteristics, improved stability and improved processing such as ease of filling into pharmaceutically acceptable capsules.

### SUMMARY OF THE INVENTION

The invention is related to a water-free liquid or semisolid pharmaceutical composition comprising a derivatized insulin peptide (a), at least one polar organic solvent (b) for the derivatized insulin peptide, at least one lipophilic component (c), and optionally at least one surfactant (d).

In one aspect the pharmaceutical composition is in the form of a clear water-free liquid.

In one aspect the pharmaceutical composition is a clear water-free liquid and comprises a derivatized insulin peptide (a), at least one polar organic solvent (b) for the derivatized insulin peptide, at least one lipophilic component (c), and optionally at least one surfactant (d).

In one aspect the pharmaceutical composition comprises at least one surfactant and the pharmaceutical composition is spontaneously dispersible.

In one aspect the pharmaceutical composition the derivatized insulin peptide is an acylated insulin peptide.

The invention also contemplates the pharmaceutical composition for use as a medicament.

### DESCRIPTION OF THE DRAWINGS

FIG. 1. Blood glucose lowering effect after oral administration (4 ml/kg) of 800 nmol/kg of the insulin derivative B29K(N(eps)Octadecanedioyl-gGlu-OEG-OEG) A14E B25H desB30 human insulin formulated in a lipid based pharmaceutical composition, (-□-) insulin dissolved in 20% propylene glycol and 80% Capmul MCM C8/10, to overnight fasted male Wistar rats (mean±SEM, n=6). A vehicle without insulin derivative was administrated as control (-▪-).

FIG. 2. Plasma exposure (in pM) of the insulin derivative B29K(N(eps)Octadecanedioyl-gGlu-OEG-OEG) A14E B25H desB30 human insulin after intestinal injection of 60 nmol/kg (0.4 ml/kg) into the mid-jejunum of fasted male SPRD rats (mean±SEM, n=5-6) formulated in different lipid based delivery systems (-▪-) 30% propylene glycol and 70% Capmul MCM C8, (-═-) 30% propylene glycol and 70% Capmul MCM C8/10, (-x-) 30% propylene glycol and 70% Capmul MCM C10, (-∘-) 30% propylene glycol and 70% Capmul PG8. The delivery system with the insulin derivative dissolved in 30% propylene glycol and 70% propylene glycol caprylate (Capmul PG8) showed highest plasma exposure.

FIG. 3. Plasma exposure (in pM) of the insulin derivative B29K(N(eps)Octadecanedioyl-gGlu-OEG-OEG) A14E B25H desB30 human insulin after intestinal injection of 60 nmol/kg (0.4 ml/kg) into the mid-jejunum of fasted male SPRD rats (mean±SEM, n=5-6) formulated in different pharmaceutical compositions (-▪-) 20% propylene glycol and 80% Capmul MCM C8/C10, (-□-) 20% propylene glycol, 50% Capmul MCM C8/10 and 30% Labrasol, (-x-) 20% propylene glycol, 50% Capmul MCM C8/C10 and 30% Chremophor RH40.

FIG. 4. Blood glucose lowering effect after oral administration (4 ml/kg) of 4800 nmol/kg of the insulin derivative B29N(eps)-hexadecandioyl-gamma-L-Glu, A14E B25H desB30 human insulin in a SEDDS (-□-) or 4800 nmol/kg B28D human insulin in SEDDS (-▪-) to overnight fasted male SPRD rats. SEDDS composition is the according insulin dissolved in 62.5% propylene glycol, 31.25% Capmul MCM 10 and 6.25% poloxamer 407 (mean±SEM, n=6). A vehicle without insulin was administrated as control (-▴-). Acylated insulin in a pharmaceutical composition as described showed a sustained blood glucose lowering effect in comparison with non acylated insulin.

FIG. 5. Plasma exposure (in pM) of the insulin derivatives A) -▴- A14E, B25H, B29K (N(eps)Octadecanedioyl-gGlu-OEG-OEG), desB30 human insulin, insulin derivative -▾- B) A14E, B16H, B25H, B29K ((N(eps)Eicosanedioyl-gGlu-[2-(2-{2-[2-(2-aminoethoxy)ethoxy]acetylamino}ethoxy)ethoxy]acetyl)), desB30 human insulin, insulin derivative -∘- C) A14E, B25H, B29K (N(eps) [2-(2-{2-[2-(2-{2-[(S)-4-carboxy-4-(19-carboxynonadecanoylamino) butyrylamino]ethoxy}ethoxy)acetylamino]ethoxy}ethoxy)acetyl]), desB30 human insulin and insulin derivative -□- D) A14E, B16H, B25H, B29K(N(eps)-[2-(2-[2-(2-[2-(Octadecandioyl-gGlu)amino]ethoxy)ethoxy]acetylamino)ethoxy]ethoxy)acetyl]), desB30 human insulin (120 nmol/kg) formulated in 15% propylene glycol, 55% Capmul MCM and 30% Labrasol after intestinal injection of 120 nmol/kg (0.4 ml/kg) into the mid-jejunum of fasted male SPRD rats (mean±SEM, n=6).

Sample preparation: Lyophilized pH neutral powder of the according insulin derivative was dissolved in propylene glycol at RT and after complete dissolution, the according lipid component and the according surfactant were added and mixed by magnetic stirring at RT for 5 to 10 minutes to result in clear homogenous liquids.

FIG. 6. Plasma exposure (in pM) of insulin derivative A) -▪- A14E, B25H, B29K (N(eps)Octadecanedioyl-gGlu-OEG-OEG), desB30 human insulin, insulin derivative -⋄- B) A14E, B16H, B25H, B29K ((N(eps)Eicosanedioyl-gGlu-[2-(2-{2-[2-(2-aminoethoxy)ethoxy]acetylamino}ethoxy)ethoxy]acetyl)), desB30 human insulin, insulin derivative -x- C) A14E, B25H, B29K (N(eps) [2-(2-{2-[2-(2-{2-[(S)-4-carboxy-4-(19-carboxynonadecanoylamino) butyrylamino]ethoxy}ethoxy)acetylamino]ethoxy}ethoxy)acetyl]), desB30 human insulin and insulin derivative -□- D) A14E, B16H, B25H, B29K(N(eps)-[2-(2-[2-(2-[2-(Octadecandioyl-gGlu)amino]ethoxy)ethoxy]acetylamino)ethoxy]ethoxy)acetyl]), desB30 human insulin (120 nmol/kg) formulated in 55% propylene glycol, 35% Capmul MCM and 10% Poloxamer 407 after intestinal injection of 120 nmol/kg (0.4 ml/kg) into the mid-jejunum of fasted male SPRD rats (mean±SEM, n=6).

Sample preparation: Lyophilized pH neutral powder of the according insulin derivative was dissolved in propylene glycol at RT and after complete dissolution, the according lipid component and the according surfactant were added and mixed by magnetic stirring at RT for 5 to 10 minutes to result in clear homogenous liquids.

FIG. 7. Plasma exposure (in pM) of insulin derivative A) -□- A14E, B25H, B29K (N(eps)Octadecanedioyl-gGlu-OEG-OEG), desB30 human insulin, insulin derivative -⋄- B) A1N-octadecandioyl-gamma-L-glutamyl-[2-(2-{2-[2-(2-aminoethoxy)ethoxy]acetylamino}ethoxy)ethoxy]acetyl A14E B25H □B29R desB30 human insulin, insulin derivative -▾- C) A14E, B25H, B29K(N(eps)Octadecandioyl-g-Glu), desB30 human Insulin, insulin derivative -▪- D) A14E, B25H, (N(eps)-[2-(2-[2-(2-[2-(Octadecandioyl-gGlu)amino]ethoxy)ethoxy]acetylamino)ethoxy]ethoxy)acetyl]), desB27, desB30 human insulin and insulin derivative -x- E) A14E, B25H, B29K(N(eps)lcosandioyl-gGlu), desB30 human insulin formulated in 55% propylene glycol, 35% Capmul MCM and 10% Poloxamer 407 after intestinal injection of 120 nmol/kg (0.4 ml/kg) into the mid-jejunum of fasted male SPRD rats (mean±SEM, n=6).

Sample preparation: Lyophilized pH neutral powder of the according insulin derivative was dissolved in propylene glycol at RT and after complete dissolution, the according lipid component and the according surfactant were added and mixed by magnetic stirring at RT for 5 to 10 minutes to result in clear homogenous liquids.

FIG. 8. Plasma exposure (in pM) of the insulin derivative B29K(N(eps)Octadecanedioyl-gGlu-OEG-OEG) A14E B25H desB30 human insulin (60 nmol/kg) dissolved in water or in propylene glycol, injected into mid-jejunum of fasted male SPRD rats (mean±SEM, n=6).

FIG. 9. Plasma exposure (in pM) of the insulin derivative B29K(N(eps)Octadecanedioyl-gGlu-OEG-OEG) A14E B25H desB30 human insulin after intestinal injection of 60 nmol/kg (0.4 ml/kg) into the mid-jejunum of fasted male SPRD rats (mean±SEM, n=5-6) formulated in different pharmaceutical compositions (-▪-) 15% propylene glycol and 40% Labrasol and 45% Rylo MG08 (glycerol caprylate), (-□-) 15% propylene glycol, 40% Labrasol, 30% Rylo MG10 (glycerol caprate) and 15% propylene glycol caprylate, (-x-) 15% propylene glycol, 40% Labrasol, 45% Rylo MG10 (glycerol caprate), and (-Δ-) 15% propylene glycol, 40% Labrasol, 30% Rylo MG08 (glycerol caprylate), 15% propylene glycol caprylate.

Sample preparation: Lyophilized pH neutral powder of the insulin derivative B29K(N(eps)Octadecanedioyl-gGlu-OEG-OEG) A14E B25H desB30 human insulin was dissolved in propylene glycol at RT and after complete dissolution, the according lipid component and the according surfactant were added and mixed by magnetic stirring at RT for 5 to 10 minutes to result in clear homogenous liquids.

FIG. 10. Blood glucose lowering effect in male beagle dogs (17 kg body weight) after peroral administration of an enteric coated HPMC capsule containing 180 nmol/kg of the insulin derivative B29K(N(eps)Octadecanedioyl-gGlu-OEG-OEG) A14E B25H desB30 human insulin (60 nmol/kg) formulated with 15% propylene glycol, 40% Labrasol and 45% Capmul MCM (Glycerol caprylate/caprate).

FIG. 11. 24 hour plasma exposure profile (in pM) of the insulin derivative B29K(N(eps)Octadecanedioyl-gGlu-OEG-OEG) A14E B25H desB30 human insulin in male beagle dogs (17 kg body weight) after peroral administration of an enteric coated soft-gelatine capsule containing 30 nmol/kg of the insulin derivative dissolved in 15% propylene glycol, 40% Labrasol and 45% Rylo MG08 Pharma (Glycerol caprylate). Soft-gelatine capsules were coated with Eudragit L 30 D-55.

### DESCRIPTION OF THE INVENTION

The present invention relates to water-free liquid or semisolid pharmaceutical compositions comprising a derivatized insulin peptide (a), at least one polar organic solvent (b) for the derivatized insulin peptide, at least one lipophilic component (c), and optionally a surfactant (d) and/or at least one solid hydrophilic component (e).

It has been found that particularly suitable water-free compositions for oral administration comprising derivatized insulin peptides, polar organic solvent(s), lipophilic component(s) and optionally surfactant(s) and/or solid hydrophilic component(s) are obtainable using a pharmaceutical composition according to the invention.

The pharmaceutical composition according to the invention has thus surprisingly been found to enhance the efficacy of uptake of said derivatized insulin peptides administered orally while providing a sustained profile of action.

Also, the derivatized insulin peptide(s) in the composition according to the invention have been found to have good stability.

In one aspect the present invention relates to pharmaceutical compositions comprising a derivatized insulin peptide (a), at least one polar organic solvent (b) for the derivatized insulin peptide, at least one lipophilic component (c), and optionally at least one solid hydrophilic component (d), wherein said pharmaceutical composition is in the form of an oily solution.

In another aspect the present invention relates to water-free liquid or semisolid pharmaceutical compositions comprising a derivatized insulin peptide (a), at least one polar organic solvent (b) for the derivatized insulin peptide, at least one lipophilic component (c), and at least one solid hydrophilic component (d), wherein said pharmaceutical composition is in the form of an oily solution. In yet another aspect the at least one solid hydrophilic component (d) is at least one solid hydrophilic polymer. In yet another aspect the pharmaceutical composition comprising at least one solid hydrophilic component is free of surfactant, wherein said surfactant has an HLB value which is at least 8, i.e. in one aspect there is no surfactant, which has an HLB value which is at least 8, present in the composition.

In one aspect the present invention relates to water-free liquid or semisolid pharmaceutical compositions comprising a derivatized insulin peptide (a), at least one polar organic solvent (b) for the derivatized insulin peptide, at least one lipophilic component (c), at least one surfactant (d) and optionally at least one solid hydrophilic component (e), wherein said pharmaceutical composition is spontaneously dispersible.

In one aspect the present invention relates to water-free liquid pharmaceutical compositions comprising a derivatized insulin peptide (a), at least one polar organic solvent (b) for the derivatized insulin peptide, at least one lipophilic component (c), and optionally at least one surfactant (d), wherein the pharmaceutical composition is in the form of a clear solution.

When the water-free liquid pharmaceutical composition is in the form of a clear solution it has the further advantage that the physical stability of the composition is improved. In one aspect of the invention the water-free liquid pharmaceutical composition according to the invention is in the form of a clear solution and is stable for more than 6 weeks of usage and for more than 3 years of storage.

In another aspect of the invention the water-free liquid pharmaceutical composition according to the invention is in the form of a clear solution and is stable for more than 4 weeks of usage and for more than 3 years of storage.

In a further aspect of the invention the water-free liquid pharmaceutical composition according to the invention is in the form of a clear solution and is stable for more than 4 weeks of usage and for more than two years of storage.

In an even further aspect of the invention the water-free liquid pharmaceutical composition according to the invention is in the form of a clear solution and is stable for more than 2 weeks of usage and for more than two years of storage.

In an even further aspect of the invention the water-free liquid pharmaceutical composition according to the invention is in the form of a clear solution and is stable for more than 1 weeks of usage and for more than one year of storage.

In one aspect the present invention relates to water-free liquid pharmaceutical compositions comprising a derivatized insulin peptide (a), at least one polar organic solvent (b) for the derivatized insulin peptide, at least one lipophilic component (c), and optionally at least one surfactant (d), wherein the pharmaceutical composition is in the form of a clear oily solution.

In one aspect the present invention relates to water-free liquid pharmaceutical compositions comprising a derivatized insulin peptide (a), at least one polar organic solvent (b) for the derivatized insulin peptide, at least one lipophilic component (c), wherein the pharmaceutical composition is in the form of a clear solution.

In one aspect of the invention all components are present as liquids or dissolved solids. The derivatized insulin peptide may thus in said aspect be dissolved in at least one polar organic solvent.

In one aspect the present invention relates to water-free liquid pharmaceutical compositions comprising a derivatized insulin peptide (a), at least one polar organic solvent (b) for the derivatized insulin peptide, at least one lipophilic component (c), and optionally at least one surfactant (d), wherein the pharmaceutical composition is in the form of a clear solution, and wherein said pharmaceutical composition is spontaneously dispersible.

In one aspect a pharmaceutical composition according to the invention is a waterfree oily solution and/or a SEDDS or SMEDDS pharmaceutical compositions.

SEDDS and SMEDDS pharmaceutical compositions according to the invention have the additional advantage of enhancing the intestinal absorption of the insulin derivative and of reducing enzymatic degradation of the insulin derivative.

In one aspect a pharmaceutical composition according to the invention is a self emulsifying drug delivery system (SEDDS). The inventor has thus found that the SEDDS according to the invention have improved oral bioavailability compared to traditional pharmaceutical compositions such as e.g. aqueous and/or lipid free polar solvent solutions often used subcutaneously.

It has been shown that the derivatized insulin peptide(s) are highly soluble in the pharmaceutically acceptable polar organic solvent of the pharmaceutical composition according to the invention. The amount of polar organic solvent needed in said pharmaceutical composition is therefore relatively low. This may improve compatibility of the pharmaceutical composition according to the invention with capsule materials.

The present invention also relates to a pharmaceutical composition that includes a derivatized insulin peptide in a carrier that comprises a lipophilic component, a surfactant and a polar organic solvent and optionally a solid hydrophilic component (e). In the aspect where there is a solid hydrophilic component present, at least one of the components selected from the group consisting of a lipophilic component and a surfactant is liquid or semi-solid. In the aspect where there is a liquid hydrophilic component (e) present, both the lipophilic component and the surfactant may be solid. In one aspect, the surfactant is liquid or semisolid. In one aspect, a solid hydrophilic component is present.

As used herein, the term “carrier” refers to the pharmaceutically acceptable vehicle that transports the therapeutically active water-soluble derivatized insulin peptide across the biological membrane or within a biological fluid. The carrier, of the present invention, comprises a lipophilic component and a polar organic solvent, and optionally a solid hydrophilic component and/or a surfactant. In one aspect the carrier comprises a lipophilic component and a polar organic solvent, and optionally a surfactant. In one aspect the carrier comprises a lipophilic component, a polar organic solvent and a surfactant. The carrier of the present invention is capable of spontaneously producing an emulsion or colloidal structures, when brought in contact, dispersed, or diluted, with an aqueous medium, e.g., water, fluids containing water, or in vivo media in mammals, such as the gastric juices of the gastrointestinal tract. The colloidal structures may be solid or liquid particles including domains, droplets, micelles, mixed micelles, vesicles and nanoparticles.

In one aspect, when the pharmaceutical composition is brought into contact with an aqueous medium, an emulsion, such as a microemulsion, spontaneously forms. In particular, an emulsion or microemulsion forms in the digestive tract of a mammal when the delivery system of the present invention is orally ingested. In addition to the aforementioned components, the spontaneously dispersible preconcentrate may also optionally contain other excipients, such as buffers, pH adjusters, stabilizers and other adjuvants recognized by one of ordinary skill in the art to be appropriate for such a pharmaceutical use.

The term “water-free” as used herein refers to a composition to which no water is added during preparation of the pharmaceutical composition. The derivatized insulin peptide and/or one or more of the excipients in the pharmaceutical composition may have small amounts of water bound to it before preparing a pharmaceutical composition according to the invention. In one aspect a water-free pharmaceutical composition according to the invention comprises less than 10% w/w water. In another aspect, the composition according to the invention comprises less than 5% w/w water. In another aspect, the composition according to the invention comprises less than 4% w/w water, in another aspect less than 3% w/w water, in another aspect less than 2% w/w water and in yet another aspect less than 1% w/w water.

As used herein, the term “microemulsion preconcentrate” means a composition, which spontaneously forms a microemulsion, e.g., an oil-in-water microemulsion, in an aqueous medium, e.g. in water or in the gastrointestinal fluids after oral application. The composition self-emulsifies upon dilution in an aqueous medium for example in a dilution of 1:5, 1:10, 1:50, 1:100 or higher.

Due to the high solubility of the derivatized insulin peptide(s) in the polar organic solvent, the total amount of polar organic solvent in the SEDDS may be kept low which on the one hand improves compatibility of the formulation with capsule materials and on the other hand gives more design space for the composition.

The pharmaceutical composition according to the invention comprises a lipophilic component and an organic polar component. The components of the drug delivery system may be present in any relative amounts. In one aspect the drug delivery system comprises up to 50% polar organic component by weight of the composition of the carrier, i.e. up to 50% of the weight of the carrier consists of the polar organic component. In one aspect the drug delivery system comprises less than 40%, 30%, 20%, 15% or 10% polar organic component by weight of the composition of the carrier. In a further aspect, the drug delivery system comprises from 5% to 40% by weight polar organic solvent of the total composition of the carrier. In yet a further aspect, the drug delivery system comprises from 10% to 30% by weight polar organic solvent of the total composition of the carrier. In one aspect, the drug delivery system comprises from 10% to 15% by weight polar organic solvent of the total composition of the carrier. In a further aspect, the drug delivery system comprises about 15% by weight polar organic solvent of the total composition of the carrier

The term “about” as used herein means in reasonable vicinity of the stated numerical value, such as plus or minus 10%.

The pharmaceutical composition according to the invention is in the form of a non-powder composition, i.e. in a semi-solid or liquid form.

In one aspect the pharmaceutical composition according to the invention is in the form of a liquid.

As used herein, the term “liquid” means a component or composition that is in a liquid state at room temperature (“RT”), and having a melting point of, for example, below 20° C. As used herein room temperature (RT) means approximately 20-25° C.

As used herein, the term “semi-solid” relates to a component or composition which is not liquid at room temperature, e.g., having a melting point between room temperature and about 40° C. A semisolid may have the qualities and/or attributes of both the solid and liquid states of matter. As used-herein, the term “solidify” means to make solid or semi-solid.

Examples of semi-solid or liquid compositions according to the invention are pharmaceutical compositions in the form of e.g. oils, solutions, liquid or semisolid SMEDDS and liquid or semisolid SEDDS.

“SMEDDS” (self-micro-emulsifying drug delivery systems) are herein defined as isotropic mixtures of a hydrophilic component, a surfactant, optionally a cosurfactant and a drug that rapidly form an oil in water microemulsion when exposed to aqueous media under conditions of gentle agitation or digestive motility that would be encountered in the GI tract.

“SEDDS” (self emulsifying drug delivery systems) are herein defined as mixtures of a hydrophilic component, a surfactant, optionally a cosurfactant and a drug that forms spontaneously a fine oil in water emulsion when exposed to aqueous media under conditions of gentle agitation or digestive motility that would be encountered in the GI tract.

As used herein, the term “microemulsion” refers to a clear or translucent, slightly opaque, opalescent, non-opaque or substantially non-opaque colloidal dispersion that is formed spontaneously or substantially spontaneously when its components are brought into contact with an aqueous medium.

As used herein, the term “emulsion” refers to a slightly opaque, opalescent or opague colloidal dispersion that is formed spontaneously or substantially spontaneously when its components are brought into contact with an aqueous medium.

A microemulsion is thermodynamically stable and contains homogenously dispersed particles or domains, for example of a solid or liquid state (e.g., liquid lipid particles or droplets), of a mean diameter of less than about 500 nm, e.g., less than about 400 nm or less than 300 nm, less than 200 nm, less than 100 nm, and greater than about 2-4 nm as measured by standard light scattering techniques, e.g., using a MALVERN ZETASIZER Nano ZS. The term “domain size” as used herein refers to repetitive scattering units and may be measured by e.g., small angle X-ray. In one aspect of the invention, the domain size is smaller than 400 nm, in another aspect, smaller than 300 nm and in yet another aspect, smaller than 200 nm.

As used herein the term “spontaneously dispersible” when referring to a pre-concentrate refers to a composition that is capable of producing colloidal structures such as microemulsions, emulsions and other colloidal systems, when diluted with an aqueous medium when the components of the composition of the invention are brought into contact with an aqueous medium, e.g. by simple shaking by hand for a short period of time, for example for ten seconds. In one aspect a spontaneously dispersible concentrate according to the invention is a SEDDS or SMEDDS.

As used herein, the term “lipophilic component” refers to a substance, material or ingredient that is more compatible with oil than with water. A material with lipophilic properties is insoluble or almost insoluble in water but is easily soluble in oil or other nonpolar solvents. The term “lipophilic component” may comprise one or more lipophilic substances. Multiple lipophilic components may constitute the lipophilic phase of the spontaneously dispersible preconcentrate and form the oil aspect, e.g., in an oil-in-water emulsion or microemulsion. At room temperature, the lipophilic component and lipophilic phase of the spontaneously dispersible preconcentrate may be solid, semisolid or liquid. For example, a solid lipophilic component may exist as a paste, granular form, powder or flake. If more than one excipient comprises the lipophilic component, the lipophilic component may be a mixture of liquids, solids, or both.

In one aspect of the invention, the lipophilic component is present in the pharmaceutical composition in an amount of at least 20% w/w of the composition of the carrier, i.e. at least 20% of the weight of the carrier consists of the lipophilic component. In a further aspect of the invention, the lipophilic component is present in an amount of at least 30%, at least 50%, at least 80% or at least 90% w/w. For example, the lipophilic component may be present from about 5% to about 90% by weight of the carrier, e.g., from about 15% to about 60%, e.g. from about 20% to about 60%, e.g. from about 20% to about 40%. In one aspect of the invention, the lipophilic component is present in an amount from 45% to 55%. In one aspect of the invention, the lipophilic component is present in an amount of about 45%.

Examples of solid lipophilic components, i.e., lipophilic components which are solid or semisolid at room temperature, include, but are not limited to, the following:

1. Mixtures of mono-, di- and triglycerides, such as hydrogenated coco-glycerides (melting point (m.p.) of about 33.5° C. to about 37° C.], commercially-available as WITEPSOL HI5 from Sasol Germany (Witten, Germany); Examples of fatty acid triglycerides e.g., C10-C22 fatty acid triglycerides include natural and hydrogenated oils, such as vegetable oils;
2. Esters, such as propylene glycol (PG) stearate, commercially available as MONOSTEOL (m.p. of about 33° C. to about 36° C.) from Gattefosse Corp. (Paramus, N.J.); diethylene glycol palmito stearate, commercially available as HYDRINE (m.p. of about 44.5° C. to about 48.5° C.) from Gattefosse Corp.;
3. Polyglycosylated saturated glycerides, such as hydrogenated palm/palm kernel oil PEG-6 esters (m.p. of about 30.5° C. to about 38° C.), commercially-available as LABRAFIL M2130 CS from Gattefosse Corp. or Gelucire 33/01;
4. Fatty alcohols, such as myristyl alcohol (m.p. of about 39° C.), commercially available as LANETTE 14 from Cognis Corp. (Cincinnati, Ohio); esters of fatty acids with fatty alcohols, e.g., cetyl palmitate (m.p. of about 50° C.); isosorbid monolaurate, e.g. commercially available under the trade name ARLAMOL ISML from Uniqema (New Castle, Del.), e.g. having a melting point of about 43° C.;
5. PEG-Fatty alcohol ether, including polyoxyethylene (2) cetyl ether, e.g. commercially available as BRIJ 52 from Uniqema, having a melting point of about 33° C., or polyoxyethylene (2) stearyl ether, e.g. commercially available as BRIJ 72 from Uniqema having a melting point of about 43° C.;
6. Sorbitan esters, e.g. sorbitan fatty acid esters, e.g. sorbitan monopalmitate or sorbitan monostearate, e.g, commercially available as SPAN 40 or SPAN 60 from Uniqema and having melting points of about 43° C. to 48° C. or about 53° C. to 57° C. and 41° C. to 54° C., respectively; and
7. Glyceryl mono-C6-C14-fatty acid esters. These are obtained by esterifying glycerol with vegetable oil followed by molecular distillation. Monoglycerides include, but are not limited to, both symmetric (i.e. β-monoglycerides) as well as asymmetric monoglycerides α-monoglycerides). They also include both uniform glycerides (in which the fatty acid constituent is composed primarily of a single fatty acid) as well as mixed glycerides (i.e. in which the fatty acid constituent is composed of various fatty acids). The fatty acid constituent may include both saturated and unsaturated fatty acids having a chain length of from e.g. C8-C14. Particularly suitable are glyceryl mono laurate e.g. commercially available as IMWITOR 312 from Sasol North America (Houston, Tex.), (m.p. of about 56° C.-60° C.); glyceryl mono dicocoate, commercially available as IMWITOR 928 from Sasol (m.p. of about 33° C.-37° C.); monoglyceryl citrate, commercially available as IMWITOR 370, (m.p. of about 59 to about 63° C.); or glyceryl mono stearate, e.g., commercially available as IMWITOR 900 from Sasol (rn.p. of about 56° C.-61° C.); or self-emulsifying glycerol mono stearate, e.g., commercially available as IMWITOR 960 from Sasol (m.p. of about 56° C.-61° C.).

Examples of liquid and semisolid lipophilic components, i.e., lipophilic components which are liquid at room temperature include, but are not limited to, the following:

1. Mixtures of mono-, di- and triglycerides, such as medium chain mono- and diglycerides, glyceryl caprylate/caprate, commercially-available as CAPMUL MCM from Abitec Corp. (Columbus, Ohio);
2. Glyceryl mono- or di fatty acid ester, e.g. of C6-C18, e.g. C6-C16 e.g. C8-C10, e.g. C8, fatty acids, or acetylated derivatives thereof, e.g. MYVACET 9-45 or 9-08 from Eastman Chemicals (Kingsport, Tenn.) or IMWITOR 308 or 312 from Sasol; Glycerol monocaprylate (such as Rylo MG08 Pharma, from Danisco) or Glycerol monocaprate (such as Rylo MG10 Pharma, from Danisco);
3. Propylene glycol mono- or di- fatty acid ester, e.g. of C8-C20, e.g. C8-C12, fatty acids, e.g. LAUROGLYCOL 90, SEFSOL 218, or CAPRYOL 90 or CAPMUL PG-8 (same as propylene glycol caprylate) from Abitec Corp.;
4. Oils, such as safflower oil, sesame oil, almond oil, peanut oil, palm oil, wheat germ oil, corn oil, castor oil, coconut oil, cotton seed oil, soybean oil, olive oil and mineral oil;
5. Fatty acids or alcohols, e.g. C8-C20, saturated or mono- or di- unsaturated, e.g. oleic acid, oleyl alcohol, linoleic acid, capric acid, caprylic acid, caproic acid, tetradecanol, dodecanol, decanol;
6. Medium chain fatty acid triglycerides, e.g. C8-C12, e.g. MIGLYOL 812, or long chain fatty acid triglycerides, e.g. vegetable oils;
7. Transesterified ethoxylated vegetable oils, e.g. commercially available as LABRAFIL M2125 CS from Gattefosse Corp;
8. Esterified compounds of fatty acid and primary alcohol, e.g. C8-C20, fatty acids and C2-C3 alcohols, e.g. ethyl linoleate, e.g. commercially available as NIKKOL VF-E from Nikko Chemicals (Tokyo, Japan), ethyl butyrate, ethyl caprylate oleic acid, ethyl oleate, isopropyl myristate and ethyl caprylate;
9. Essential oils, or any of a class of volatile oils that give plants their characteristic odors, such as spearmint oil, clove oil, lemon oil and peppermint oil;
10. Fractions or constituents of essential oils, such as menthol, carvacrol and thymol;
11. Synthetic oils, such as triacetin, tributyrin;
12. Triethyl citrate, acetyl triethyl citrate, tributyl citrate, acetyl tributyl citrate;
13. Polyglycerol fatty acid esters, e.g. diglyceryl monooleate, e.g. DGMO-C, DGMO-90, DGDO from Nikko Chemicals; and
14. Sorbitan esters, e.g. sorbitan fatty acid esters, e.g. sorbitan monolaurate, e.g. commercially available as SPAN 20 from Uniqema.
15. Phospholipids, e.g. Alkyl-O-Phospholipids, Diacyl Phosphatidic Acids, Diacyl Phosphatidyl Cholines, Diacyl Phosphatidyl Ethanolamines, Diacyl Phosphatidyl Glycerols, Di-O-Alkyl Phosphatidic Acids, L-alpha-Lysophosphatidylcholines (LPC), L-alpha-Lysophosphatidylethanolamines (LPE), L-alpha-Lysophosphatidylglycerol (LPG), L-alpha-Lysophosphatidylinositols (LPI), L-alpha-Phosphatidic acids (PA), L-alpha-Phosphatidylcholines (PC), L-alpha-Phosphatidylethanolamines (PE), L-alpha-Phosphatidylglycerols (PG), Cardiolipin (CL), L-alpha-Phosphatidylinositols (PI), L-alpha-Phosphatidylserines (PS), Lyso-Phosphatidylcholines, Lyso-Phosphatidylglycerols, sn-Glycerophosphorylcholines commercially available from LARODAN, or soybean phospholipid (Lipoid S100) commercially available from Lipoid GmbH.

In one aspect of the invention, the lipophilic component is one or more selected from the group consisting of mono-, di-, and triglycerides. In a further aspect, the lipophilic component is one or more selected from the group consisting of mono- and diglycerides. In yet a further aspect, the lipophilic component is Capmul MCM or Capmul PG-8. In a still further aspect, the lipophilic component is Capmul PG-8. In yet another aspect, the lipophilic component is glycerol monocaprylate (e.g. Rylo MG08 Pharma from Danisco).

The term “polar organic solvent” refers in one aspect herein to a “polar protic organic solvent” which is a hydrophilic, water miscible carbon-containing solvent that contains an O—H or N—H bond, or mixtures thereof. The polarity is reflected in the dielectric constant or the dipole moment of a solvent. The polarity of a solvent determines what type of compounds it is able to dissolve and with what other solvents or liquid compounds it is miscible. Typically, polar organic solvents dissolve polar compounds best and non-polar solvents dissolve non-polar compounds best: “like dissolves like”. Strongly polar compounds like inorganic salts (e.g. sodium chloride) dissolve only in very polar solvents.

Polar organic solvents of the invention may be selected from solvents wherein derivatized insulin peptides show better solubility in said polar organic solvents than in other solvents.

It has thus been found that derivatized insulin peptides such as acylated insulin peptides can be dissolved to a high degree in a water-free pharmaceutical acceptable polar organic solvent such as propylene glycol, glycerol and PEG200. In one aspect at least 20% (w/w) of the derivatized insulin peptides dissolve in a water-free pharmaceutical acceptable polar organic solvent according to the invention, i.e. when adding 20% w/w derivatized insulin peptide to the polar organic solvent a clear solution is obtained. In another aspect at least 25%, 30%, 40% or 50% (w/w) of the derivatized insulin peptides dissolve in a water-free pharmaceutical acceptable polar organic solvent according to the invention.

The polar organic solvent may thus refer to a hydrophilic, water miscible carbon-containing solvent that contains an O—H or N—H bond, or mixtures thereof. The polarity is reflected in the dielectric constant or the dipole moment of a solvent. The polarity of a solvent determines what type of compounds it is able to dissolve and with what other solvents or liquid compounds it is miscible. Typically, polar solvents dissolve polar compounds best and non-polar solvents dissolve non-polar compounds best: “like dissolves like”. Strongly polar compounds like inorganic salts (e.g. sodium chloride) dissolve only in very polar solvents.

In a further aspect of the invention, the polar organic solvent is a solvent having a dielectric constant above 20, preferably in the range of 20-50. Examples of different polar organic solvent are listed in Table 1 together with water as a reference.

TABLE 1
Dielectric constants (static permittivity) of selected polar organic solvents
and water as a reference (Handbook of Chemistry and Physics,
CMC Press, dielectric constants are measured in static electric
fields or at relatively low frequencies, where no relaxation occurs)

| Solvent (Temperature, Kelvin) | Dielectric constant, ∈* |
|---|---|
| Water (293.2) | 80.1 |
| Propanetriol [Glycerol] (293.2) | 46.53 |
| Ethanediol [Ethylene Glycol] (293.2) | 41.4 |
| 1,3-propanediol (293.2) | 35.1 |
| Methanol (293.2) | 33.0 |
| 1,4-butanediol (293.2) | 31.9 |
| 1,3-butanediol (293.2) | 28.8 |
| 1,2-propanediol [propylene glycol] (303.2) | 27.5 |
| Ethanol (293.2) | 25.3 |
| Isopropanol (293.2) | 20.18 |

In the present context, 1,2-propanediol and propylene glycol is used interchangeably. In the present context, propanetriol and glycerol is used interchangeably. In the present context, ethanediol and ethylene glycol is used interchangeably.

In one aspect of the invention, the polar organic solvent is selected from the group consisting of polyols. The term “polyol” as used herein refers to chemical compounds containing multiple hydroxyl groups.

In a further aspect of the invention, the polar organic solvent is selected from the group consisting of diols and triols. The term “diol” as used herein refers to chemical compounds containing two hydroxyl groups. The term “triol” as used herein refers to chemical compounds containing three hydroxyl groups.

In a further aspect of the invention, the polar organic solvent is selected from the group consisting of glycerol (propanetriol), ethanediol (ethylene glycol), 1,3-propanediol, methanol, 1,4-butanediol, 1,3-butanediol, propylene glycol (1,2-propanediol), ethanol and isopropanol, or mixtures thereof. In a further aspect of the invention, the polar organic solvent is selected from the group consisting of propylene glycol and glycerol. In another aspect of the invention, the polar organic solvent is glycerol. This polar organic solvent is biocompatible even at high dosages and has a high solvent capacity for e.g. insulin peptides and GLP-1 compounds. In another aspect of the invention, the polar organic solvent is selected from the group consisting of propylene glycol and ethylene glycol. These polar organic solvent have a low viscosity, are biocompatible at moderate doses, and have very high polar organic solvent t capacity for e.g. insulin peptides and GLP-1 compounds. In another aspect of the invention, the polar organic solvent is propylene glycol.

The polar organic solvent should preferably be of high purity with a low content of e.g. aldehydes, ketones and other reducing impurities in order to minimize chemical deterioration of the solubilized derivatized insulin peptide due to e.g. Maillard reaction. Scavenger molecules like glycyl glycine and ethylene diamine may be added to the formulations comprising polar organic solvent (s) such as polyols to reduce deterioration of the derivatized insulin peptide whereas antioxidants may be added to reduce the rate of formation of further reducing impurities.

In one aspect of the invention, the polar organic solvent is present in the pharmaceutical composition in an amount of 1-50% w/w by weight of the composition of the carrier, i.e. from 1% to 50% of the weight of the carrier consists of the polar organic component. In a further aspect of the invention, the polar organic solvent is present in an amount of 5-40% w/w. In a further aspect of the invention, the polar organic is present in an amount of 5-30% w/w. In a further aspect of the invention, the organic polar solvent is present in an amount of 10-30% w/w. In a further aspect of the invention, the polar organic solvent is present in an amount of 10-25% w/w. In a further aspect of the invention, the polar organic solvent is present in an amount of 10-15% w/w. In a further aspect of the invention, the polar organic solvent is present in an amount of about 20% w/w. In a further aspect of the invention, the polar organic solvent is present in an amount of about 15% w/w.

In one aspect of the invention, the polar organic polar solvent is propylene glycol and is present in the carrier of the pharmaceutical composition in an amount of 1-50% w/w. In a further aspect of the invention, propylene glycol is present in an amount of 5-40% w/w. In a further aspect of the invention, propylene glycol is present in an amount of 10-30% w/w. In a further aspect of the invention, propylene glycol is present in an amount of 10-25% w/w. In a further aspect of the invention, propylene glycol is present in an amount of 10-20% w/w. In a further aspect of the invention, propylene glycol is present in an amount of 10-15% w/w. In a further aspect of the invention, propylene glycol is present in an amount of about 20% w/w. In a further aspect of the invention, propylene glycol is present in an amount of about 15% w/w.

In one aspect of the invention, the polar organic solvent is selected from the group consisting of glycerol, propylene glycol and mixtures thereof. In a further aspect, the polar organic solvent is glycerol. In a further aspect, the polar organic solvent is a mixture of glycerol and propylene glycol. In yet a further aspect, the polar organic solvent is propylene glycol.

A solid hydrophilic component may be added to the pharmaceutical composition in order to render or help render the pharmaceutical composition solid or semi-solid at room temperature. The hydrophilic component may comprise more than one excipient. If more than one excipient comprises the hydrophilic component, the hydrophilic component may be a mixture of liquids, solids, or both.

When a solid hydrophilic component is present, the carrier of the pharmaceutical composition may comprise from about 1% to about 25% by weight of solid hydrophilic component, e.g., from about 2% to about 20%, e.g., from about 3% to about 15%, e.g. from about 4% to about 10%.

An example of a hydrophilic component is PEG which is the polymer of ethylene oxide that conforms generally to the formula H(OCH₂CH₂)ₙ0H in which n correlates with the average molecular weight of the polymer.

The types of PEG useful in the present invention may be categorized by its state of matter, i.e., whether the substance exists in a solid or liquid form at room temperature and pressure. As used herein, “solid PEG” refers to PEG having a molecular weight such that the substance is in a solid state at room temperature and pressure. For example, PEG having a molecular weight ranging between 1,000 and 10,000 is a solid PEG. Such PEGs include, but are not limited to PEG 1000, PEG 1550, PEG 2000, PEG 3000, PEG 3350, PEG 4000 or PEG 8000. Particularly useful solid PEGs are those having a molecular weight between 1,450 and 8,000. Especially useful as a solid PEG are PEG 1450, PEG 3350, PEG 4000, PEG 8000, derivatives thereof and mixtures thereof. PEGs of various molecular weights are commercially-available as the CARBOWAX SENTRY series from Dow Chemicals (Danbury, Conn.). Moreover, solid PEGs have a crystalline structure, or polymeric matrix, which is a particularly useful attribute in the present invention, Polyethylene oxide (“PEO”) which has an identical structure to PEG but for chain length and end groups are also suitable for use in the present invention. Various grades of PEO are commercially available as POLYOX from Dow Chemicals. PEO, for example, has a molecular weight ranging from about 100,000 to 7,000,000. The hydrophilic component in the present invention may comprise PEG, PEO, and any combinations of the foregoing.

The hydrophilic components of the present invention may optionally include a lower alkanol, e.g., ethanol. While the use of ethanol is not essential, it may improve solubility of the derivatized insulin peptide in the carrier, improve storage characteristics and/or reduce the risk of drug precipitation.

In an alternative exemplary aspect, the hydrophilic component of the carrier consists of a single hydrophilic component, e.g., a solid PEG, e.g., PEG 1450, PEG 3350, PEG 4000 and PEG 8000. In this exemplary aspect, the hydrophilic phase of the microemulsion component consists of a single hydrophilic substance. For example, if the carrier comprised PEG 3350, the carrier would contain no other hydrophilic substances, e.g., lower alkanols (lower alkyl being C₁-C₄), such as ethanol; or water.

In yet another alternative exemplary aspect, the hydrophilic component of the carrier consists of a mixture of solid PEGs. For example, the hydrophilic component comprises PEG 1450, PEG 3350, PEG 4000, PEG 8000, derivatives thereof and any combinations and mixtures thereof.

In one aspect the carrier comprises one or more surfactants, i.e., optionally a mixture of surfactants; or surface active agents, which reduce interfacial tension. The surfactant is e.g., nonionic, ionic or amphoteric. Surfactants may be complex mixtures containing side products or un-reacted starting products involved in the preparation thereof, e.g., surfactants made by polyoxyethylation may contain another side product, e.g., PEG. The surfactant or surfactants according to the invention have a hydrophilic-lipophilic balance (HLB) value which is at least 8. For example, the surfactant may have a mean HLB value of 8-30, e.g., 12-30, 12-20 or 13-15. The surfactants may be liquid, semisolid or solid in nature.

The Hydrophilic-lipophilic balance (HLB) of a surfactant is a measure of the degree to which it is hydrophilic or lipophilic, determined by calculating values for the different regions of the molecule, as described by Griffin (Griffin WC: “Classification of Surface-Active Agents by ‘HLB,’” Journal of the Society of Cosmetic Chemists 1 (1949): 311) or by Davies (Davies JT: “A quantitative kinetic theory of emulsion type, I. Physical chemistry of the emulsifying agent,” Gas/Liquid and Liquid/Liquid Interface. Proceedings of the International Congress of Surface Activity (1957): 426-438).

The term “surfactant” as used herein refers to any substance, in particular a detergent that may adsorb at surfaces and interfaces, like liquid to air, liquid to liquid, liquid to container or liquid to any solid. The surfactant may be selected from a detergent, such as caprylocaproyl macrogol-8 glycerides (such as Labrasol from Gattefosse), ethoxylated castor oil, polyglycolyzed glycerides, acetylated monoglycerides, sorbitan fatty acid esters, polysorbate, such as polysorbate-20, poloxamers, such as poloxamer 188 and poloxamer 407, polyoxyethylene sorbitan fatty acid esters, polyoxyethylene derivatives such as alkylated and alkoxylated derivatives (tweens, e.g. Tween-20, or Tween-80), monoglycerides or ethoxylated derivatives thereof, diglycerides or polyoxyethylene derivatives thereof, glycerol, cholic acid or derivatives thereof, lecithins, alcohols and phospholipids, glycerophospholipids (lecithins, cephalins, phosphatidyl serine), glyceroglycolipids (galactopyransoide), sphingophospholipids (sphingomyelin), and sphingoglycolipids (ceramides, gangliosides), DSS (docusate sodium, CAS registry no [577-11-7]), docusate calcium, CAS registry no [128-49-4]), docusate potassium, CAS registry no [749]-09-0]), SDS (sodium dodecyl sulfate or sodium lauryl sulfate), dipalmitoyl phosphatidic acid, sodium caprylate, bile acids and salts thereof and glycine or taurine conjugates, ursodeoxycholic acid, sodium cholate, sodium deoxycholate, sodium taurocholate, sodium glycocholate, N-hexadecyl-N,N-dimethyl-3-ammonio-1-propanesulfonate, anionic (alkyl-aryl-sulphonates) monovalent surfactants, palmitoyl lysophosphatidyl-L-serine, lysophospholipids (e.g. 1-acyl-sn-glycero-3-phosphate esters of ethanolamine, choline, serine or threonine), alkyl, alkoxyl (alkyl ester), alkoxy (alkyl ether)- derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the postively charged DODAC, DOTMA, DCP, BISHOP, lysophosphatidylserine and lysophosphatidylthreonine, zwitterionic surfactants (e.g. N-alkyl-N,N-dimethylammonio-1-propanesulfonates, 3-cholamido-1-propyldimethylammonio-1-propanesulfonate, dodecyl-phosphocholine, myristoyl lysophosphatidylcholine, hen egg lysolecithin), cationic surfactants (quaternary ammonium bases) (e.g. cetyl-trimethylammonium bromide, cetylpyridinium chloride), non-ionic surfactants (e.g. alkyl glucosides like dodecyl β-D-glucopyranoside, dodecyl β-D-maltoside, tetradecyl β-D-glucopyranoside, decyl β-D-maltoside, dodecyl β-D-maltoside, tetradecyl β-D-maltoside, hexadecyl β-D-maltoside, decyl β-D-maltotrioside, dodecyl β-D-maltotrioside, tetradecyl β-D-maltotrioside, hexadecyl β-D-maltotrioside, n-dodecyl-sucrose, n-decyl-sucrose, fatty alcohol ethoxylates (e.g. polyoxyethylene alkyl ethers like octaethylene glycol mono tridecyl ether, octaethylene glycol mono dodecyl ether, octaethylene glycol mono tetradecyl ether), block copolymers as polyethyleneoxide/polypropyleneoxide block copolymers (Pluronics/Tetronics, Triton X-100) ethoxylated sorbitan alkanoates surfactants (e.g. Tween-40, Tween-80, Brij-35), fusidic acid derivatives (e.g. sodium tauro-dihydrofusidate etc.), long-chain fatty acids and salts thereof C8-C20 (eg. oleic acid and caprylic acid), acylcarnitines and derivatives, N-acylated derivatives of lysine, arginine or histidine, or side-chain acylated derivatives of lysine or arginine, N-acylated derivatives of dipeptides comprising any combination of lysine, arginine or histidine and a neutral or acidic amino acid, N-acylated derivative of a tripeptide comprising any combination of a neutral amino acid and two charged amino acids, or the surfactant may be selected from the group of imidazoline derivatives, or mixtures thereof.

Examples of solid surfactants include, but are not limited to,

1. Reaction products of a natural or hydrogenated castor oil and ethylene oxide. The natural or hydrogenated castor oil may be reacted with ethylene oxide in a molar ratio of from about 1:35 to about 1:60, with optional removal of the PEG component from the products. Various such surfactants are commercially available, e-g., the CREMOPHOR series from BASF Corp. (Mt. Olive, N.J.), such as CREMOPHOR RH 40 which is PEG40 hydrogenated castor oil which has a saponification value of about 50- to 60, an acid value less than about one, a water content, i.e., Fischer, less than about 2%, an n_{D}⁶⁰ of about 1.453-1.457, and an HLB of about 14-16;
2. Polyoxyethylene fatty acid esters that include polyoxyethylene stearic acid esters, such as the MYRJ series from Uniqema e.g., MYRJ 53 having a m.p. of about 47° C.
Particular compounds in the MYRJ series are, e.g., MYRJ 53 having an m.p. of about 47° C. and PEG-40-stearate available as MYRJ 52;
3. Sorbitan derivatives that include the TWEEN series from Uniqema, e.g., TWEEN 60;
4. Polyoxyethylene-polyoxypropylene co-polymers and block co-polymers or poloxamers, e.g., Pluronic F127, Pluronic F68 from BASF;
5. Polyoxyethylene alkyl ethers, e.g., such as polyoxyethylene glycol ethers of C₁₂-C₁₈ alcohols, e.g., polyoxyl 10- or 20-cetyl ether or polyoxyl 23-lauryl ether, or 20-oleyl ether, or polyoxyl 10-, 20- or 100-stearyl ether, as known and commercially available as the BRIJ series from Uniqema. Particularly useful products from the BRIJ series are BRIJ 58; BRIJ 76; BRIJ 78; BRIJ 35, i.e. polyoxyl 23 lauryl ether; and BRIJ 98, i.e., polyoxyl 20 oleyl ether. These products have a m.p. between about 32° C. to about 43° C.;
6. Water-soluble tocopheryl PEG succinic acid esters available from Eastman Chemical Co. with a m.p. of about 36° C., e.g., TPGS, e.g., vitamin E TPGS.
7. PEG Sterol ethers having, e.g., from 5-35 [CH₂—CH, —O] units, e.g., 20-30 units, e-g., SOLULAN C24 (Choleth-24 and Cetheth-24) from Chemron (Paso Robles, Calif.); similar products which may also be used are those which are known and commercially available as NIKKOL BPS-30 (polyethoxylated 30 phytosterol) and NIKKOL BPSH-25 (polyethoxylated 25 phytostanol) from Nikko Chemicals;
8. Polyglycerol fatty acid esters, e.g., having a range of glycerol units from 4-10, or 4, 6 or 10 glycerol units. For example, particularly suitable are deca-/hexa-/tetraglyceryl monostearate, e.g., DECAGLYN, HEXAGLYN and TETRAGLYN from Nikko Chemicals;
9. Alkylene polyol ether or ester, e.g., lauroyl macrogol-32 glycerides and/or stearoyl macrogol-32 glycerides which are GELUCIRE 44/14 and GELUCIRE 50/13 respectively;
10. Polyoxyethylene mono esters of a saturated C₁₀ to C₂₂, such as C₁₈ substituted e.g. hydroxy fatty acid; e.g. 12 hydroxy stearic acid PEG ester, e.g. of PEG about e.g. 600-900 e.g. 660 Daltons MW, e.g. SOLUTOL HS 15 from BASF (Ludwigshafen, 20 Germany). According to a BASF technical leaflet MEF 151E (1986), SOLUTOL HS 15 comprises about 70% polyethoxylated 12-hydroxystearate by weight and about 30% by weight unesterified polyethylene glycol component. It has a hydrogenation value of 90 to 110, a saponification value of 53 to 63, an acid number of maximum 1, and a maximum water content of 0.5% by weight;
11. Polyoxyethylene-polyoxypropylene-alkyl ethers, e.g. polyoxyethylene-polyoxypropylene-ethers of C₁₂ to C₁₈ alcohols, e.g. polyoxyethylen-20-polyoxypropylene-4-cetylether which is commercially available as NIKKOL PBC 34 from Nikko Chemicals;
12. Polyethoxylated distearates, e.g. commercially available under the tradenames ATLAS G 1821 from Uniqema and NIKKOCDS-6000P from Nikko Chemicals; and
13. Lecithins, e.g. soy bean phospholipid, e.g. commercially available as LIPOID S75 from Lipoid GmbH (Ludwigshafen, Germany) or egg phospholipid, commercially available as PHOSPHOLIPON 90 from Nattermann Phospholipid (Cologne, Germany).

Examples of liquid surfactants include, but are not limited to, sorbitan derivatives such as TWEEN 20, TWEEN 40 and TWEEN 80, SYNPERONIC L44, and polyoxyl 10-oleyl ether, all available from Uniqema, and polyoxyethylene containing surfactants e.g. PEG-8 caprylic/capric glycerides (e.g. Labrasol available from Gattefosse).

The carrier of the pharmaceutical composition of the invention may comprise from about 0% to about 95% by weight surfactant , e.g. from about 5% to about 80% by weight, e.g., about 10% to about 70% by weight, e.g. from about 20% to about 60% by weight, e.g. from about 30% to about 50%. In one aspect of the invention, the carrier comprises from 30 to 40% w/w surfactant. In one aspect of the invention, the carrier comprises about 40% w/w surfactant.

In one aspect of the invention, the surfactant is polyoxyethylene-polyoxypropylene co-polymers and block co-polymers or poloxamers, e.g., Pluronic F127, Pluronic F68 from BASF.

In one aspect of the invention, the surfactant is a poloxamer. In a further aspect, the surfactant is selected from the group consisting of poloxamer 188, poloxamer 407 and mixtures of poloxamer 407 and poloxamer 188.

In one aspect of the invention, the surfactant is a polyoxyethylene containing surfactants e.g. PEG-8 caprylic/capric glycerides (e.g. caprylocaproyl macrogol-8 glycerides such as Labrasol available from Gattefosse).

In one aspect of the invention, the surfactant is a lauroyl polyoxylglyceride (e.g. Gelucire 44/14 available from Gattefosse).

In one aspect of the invention, the surfactant is Cremophor RH40 from BASF.

In certain aspects of the present invention, the pharmaceutical composition may comprise additional excipients commonly found in pharmaceutical compositions, examples of such excipients include, but are not limited to, antioxidants, antimicrobial agents, enzyme inhibitors, stabilizers, preservatives, flavors, sweeteners and other components as described in Handbook of Pharmaceutical Excipients, Rowe et al., Eds., 4′h Edition, Pharmaceutical Press (2003), which is hereby incorporated by reference.

These additional excipients may be in an amount from about 0.05-5% by weight of the total pharmaceutical composition. Antioxidants, anti-microbial agents, enzyme inhibitors, stabilizers or preservatives typically provide up to about 0.05-1% by weight of the total pharmaceutical composition. Sweetening or flavoring agents typically provide up to about 2.5% or 5% by weight of the total pharmaceutical composition.

Examples of antioxidants include, but are not limited to, ascorbic acid and its derivatives, tocopherol and its derivatives, butyl hydroxyl anisole and butyl hydroxyl toluene.

In one aspect of the invention, the composition comprises a buffer. The term “buffer” as used herein refers to a chemical compound in a pharmaceutical composition that reduces the tendency of pH of the composition to change over time as would otherwise occur due to chemical reactions. Buffers include chemicals such as sodium phosphate, TRIS, glycine and sodium citrate.

The term “preservative” as used herein refers to a chemical compound which is added to a pharmaceutical composition to prevent or delay microbial activity (growth and metabolism). Examples of pharmaceutically acceptable preservatives are phenol, m-cresol and a mixture of phenol and m-cresol.

The term “stabilizer” as used herein refers to chemicals added to peptide containing pharmaceutical compositions in order to stabilize the peptide, i.e. to increase the shelf life and/or in-use time of such compositions. Examples of stabilizers used in pharmaceutical formulations are L-glycine, L-histidine, arginine, glycylglycine, ethylenediamine, citrate, EDTA, zinc, sodium chloride, polyethylene glycol, carboxymethylcellulose, and surfactants and antioxidants like alfa-tocopherol and I-ascorbic acid.

In a further aspect of the present invention, a process for preparing a pharmaceutical composition containing a derivatized insulin peptide according to the invention comprises the steps of bringing the drug and a carrier comprising a polar organic solvent, a lipophilic component, and optionally a surfactant and/or a hydrophilic component into intimate admixture. For example, the derivatized insulin peptide and the carrier may be liquefied, for example, by heating to about 20° C. to about 80° C., and then solidified by cooling to room temperature.

The carrier comprising a polar organic solvent, a lipophilic component, and optionally a surfactant and/or a hydrophilic component may be prepared separately before bringing the carrier into intimate admixture with the derivatized insulin peptide. Alternatively, one, two or more of the components of the carrier may be mixed together with the derivatized insulin peptide.

The derivatized insulin peptide may be dissolved in the polar organic solvent, and then be mixed with the lipid component and optionally with a surfactant.

In yet a further aspect, the invention provides a process for preparing a pharmaceutical composition such as SEDDS or SMEDDS (which may be filled into a capsule, e.g. enteric coated capsule, soft capsule or enteric soft capsule) containing a derivatized insulin peptide, which process comprises the following steps:

(a) dissolving first the derivatized insulin peptide in the polar organic solvent (such as propylene glycol) and
(b) then mixing with the lipophilic component, surfactant and optionally additional components.

In one aspect of the present invention, a process for preparing the pharmaceutical composition is carried out at low temperature (e.g. room temperature or below room temperature).

When preparing the pharmaceutical composition according to the invention, the derivatized insulin peptide may e.g. be dissolved in the polar organic solvent using the following method:
a) providing an aqueous solution of the derivatized insulin peptide optionally comprising excipients,
b) adjusting the pH value to a target pH value which is 1 unit, alternatively 2 units and alternatively 2.5 pH units above or below the pI of the derivatized insulin peptide,
c) removing water (dehydrating) the derivatized insulin peptide by conventional drying technologies such as freeze- and spray drying, and
d) mixing and dissolving the derivatized insulin peptide in said polar non-aqueous solvent e.g. by stirring, tumbling or other mixing methods,
e) optionally filtering or centrifuging the non-aqueous derivatized insulin peptide solution to remove non-dissolved inorganic salts,
f) optionally removing residual amounts of waters by e.g. adding solid dessicants or vacuum drying.

In one aspect the derivatized insulin peptide is dissolved in the polar organic solvent by the following method:
a) providing an aqueous solution of a derivatized insulin peptide, optionally containing stabilizers such as zinc and glycylglycine,
b) adjusting the pH value to 1 unit, alternatively 2 units and alternatively 2.5 pH units above or below the pI of the derivatized insulin peptide e.g. by adding a non-volatile base or a acid, such as hydrochloric acid or sodium hydroxide, to the solution
c) removing water from (dehydrating) the derivatized insulin peptide by conventional drying technologies such as freeze- and spray drying,
d) mixing and dissolving of the derivatized insulin peptide in said polar non-aqueous solvent e.g. by stirring, tumbling or other mixing methods,
e) optionally filtering or centrifuging the non-aqueous derivatized insulin peptide solution to remove non-dissolved inorganic salts,
f) optionally removing residual amounts of waters by e.g. adding solid dessicants or vacuum drying.

By “volatile base” is meant a base, which to some extend will evaporate upon heating and/or at reduced pressure, e.g. bases which have a vapour pressure above 65 Pa at room temperature or an aqueous azeotropic mixture including a base having a vapour pressure above 65 Pa at room temperature. Examples of volatile bases are ammonium hydroxides, tetraalkylammonium hydroxides, secondary amines, tertiary amines, aryl amines, alphatic amines or ammonium bicarbonate or a combination. For example the volatile base may be bicarbonate, carbonate, ammonia, hydrazine or an organic base such as a lower aliphatic amines e.g. trimethyl amine, triethylamine, diethanolamines, triethanolamine and their salts. Further the volatile base may be ammonium hydroxide, ethyl amine or methyl amine or a combination hereof.

By “volatile acid” is meant an acid, which to some extend will evaporate upon heating and/or at reduced pressure, e.g. acids which have a vapour pressure above 65 Pa at room temperature or an aqueous azeotropic mixture including an acid having a vapour pressure above 65 Pa at room temperature. Examples of volatile acids are carbonic acid, formic acid, acetic acid, propionic acid and butyric acid.

A “non volatile base” as mentioned herein means a base, which does not evaporate or only partly evaporate upon heating, e.g. bases with a vapour pressure below 65 Pa at room temperature. The non volatile base may be selected from the group consisting of alkaline metal salts, alkaline metal hydroxides, alkaline earth metal salts, alkaline earth metal hydroxides and amino acids or a combination hereof. Examples of non-volatile bases are sodium hydroxide, potassium hydroxide, calcium hydroxide, and calcium oxide.

A “non volatile acid” as mentioned herein means an acid, which does not evaporate or only partly evaporate upon heating, e.g. bases with a vapour pressure below 65 Pa at room temperature. Examples of non-volatile acids are hydrochloric acid, phosphoric acid and sulfuric acid.

The term “therapeutically active derivatized insulin peptide” or “therapeutic derivatized insulin peptides” as used herein refers to a derivatized insulin peptide able to cure, alleviate or partially arrest the clinical manifestations of diabetes and/or hyperglycemia and the complications therefrom.

In a further aspect of the invention, the term “therapeutically active derivatized insulin peptide” or “therapeutic derivatized insulin peptides” as used herein means a derivatized insulin peptide which is being developed for therapeutic use, or which has been developed for therapeutic use.

An amount adequate to accomplish this is defined as “therapeutically effective amount”.

Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

The therapeutically active derivatized insulin peptide may be present in an amount up to about 40% such as up to about 20% by weight of the total pharmaceutical composition, or from about 0.01% such as from about 0.1%. In one aspect of the invention, the therapeutically active derivatized insulin peptide may be present in an amount from about 0.01% to about 30%, in a further aspect from about 0.01% to 20%, 0.1% to 30%, 1% to 20% or from about 1% to 10% by weight of the total composition. It is intended, however, that the choice of a particular level of derivatized insulin peptide will be made in accordance with factors well-known in the pharmaceutical arts, including the solubility of the derivatized insulin peptide in the polar organic solvent or optional hydrophilic component or surfactant used, or a mixture thereof, mode of administration and the size and condition of the patient.

The term “pharmaceutically acceptable” as used herein means suited for normal pharmaceutical applications, i.e. giving rise to no serious adverse events in patients etc.

The term “treatment of a disease” as used herein means the management and care of a patient having developed the disease, condition or disorder. The purpose of treatment is to combat the disease, condition or disorder. Treatment includes the administration of the active compounds to eliminate or control the disease, condition or disorder as well as to alleviate the symptoms or complications associated with the disease, condition or disorder, and prevention of the disease, condition or disorder.

The term “prevention of a disease” as used herein is defined as the management and care of an individual at risk of developing the disease prior to the clinical onset of the disease. The purpose of prevention is to combat the development of the disease, condition or disorder, and includes the administration of the active compounds to prevent or delay the onset of the symptoms or complications and to prevent or delay the development of related diseases, conditions or disorders.

Each unit dosage will suitably contain from 0.1 mg to 300 mg derivatized insulin peptide, e.g. about 0.1 mg, 1 mg, 5 mg, 10 mg, 15 mg, 25 mg, 50 mg, 90 mg, 100 mg, 200 mg, 250 mg, 300 mg derivatized insulin peptide, e.g. between 5 mg and 300 mg of derivatized insulin peptide. In one aspect of the invention each unit dosage contains between 10 mg and 300 mg of derivatized insulin peptide. In a further aspect a unit dosage form contains between 10 mg and 100 mg of derivatized insulin peptide. In yet a further aspect of the invention, the unit dosage form contains between 20 mg and 300 mg of derivatized insulin peptide. In yet a further aspect of the invention, the unit dosage form contains between 50 mg and 150 mg of derivatized insulin peptide. In yet a further aspect of the invention, the unit dosage form contains between 20 mg and 100 mg of derivatized insulin peptide. Such unit dosage forms are suitable for administration 1-5 times daily depending upon the particular purpose of therapy.

The term “polypeptide” or “peptide” is used interchangeably herein to mean a compound composed of at least five constituent amino acids connected by peptide bonds. The constituent amino acids may be from the group of the amino acids encoded by the genetic code and they may be natural amino acids which are not encoded by the genetic code, as well as synthetic amino acids. Natural amino acids which are not encoded by the genetic code are e.g. hydroxyproline, γ-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (α-aminoisobutyric acid), Abu (α-aminobutyric acid), Tle (tert-butylglycine), β-alanine, 3-aminomethyl benzoic acid, anthranilic acid.

With “insulin peptide” as used herein is meant human insulin, porcine insulin or bovine insulin with disulfide bridges between CysA7 and CysB7 and between CysA20 and CysB19 and an internal disulfide bridge between CysA6 and CysA11 or an insulin analogue or derivative thereof.

Human insulin consists of two polypeptide chains, the A and B chains which contain 21 and 30 amino acid residues, respectively. The A and B chains are interconnected by two disulphide bridges. Insulin from most other species is similar, but may contain amino acid substitutions in some positions.

An insulin analogue as used herein is a polypeptide which has a molecular structure which formally can be derived from the structure of a naturally occurring insulin, for example that of human insulin, by deleting and/or substituting at least one amino acid residue occurring in the natural insulin and/or by adding at least one amino acid residue.

In one aspect an insulin analogue according to the invention comprises less than 8 modifications (substitutions, deletions, additions) relative to human insulin. In one aspect an insulin analogue comprises less than 7 modifications (substitutions, deletions, additions) relative to human insulin. In one aspect an insulin analogue comprises less than 6 modifications (substitutions, deletions, additions) relative to human insulin. In another aspect an insulin analogue comprises less than 5 modifications (substitutions, deletions, additions) relative to human insulin. In another aspect an insulin analogue comprises less than 4 modifications (substitutions, deletions, additions) relative to human insulin. In another aspect an insulin analogue comprises less than 3 modifications (substitutions, deletions, additions) relative to human insulin. In another aspect an insulin analogue comprises less than 2 modifications (substitutions, deletions, additions) relative to human insulin.

A derivatized insulin peptide according to the invention is a naturally occurring insulin or an insulin analogue which has been chemically modified, e.g. by introducing a side chain in one or more positions of the insulin backbone or by oxidizing or reducing groups of the amino acid residues in the insulin or by converting a free carboxylic group to an ester group or to an amide group. Other derivatives are obtained by acylating a free amino group or a hydroxy group, such as in the B29 position of human insulin or desB30 human insulin. A non-limiting example of acylated polypeptides may e.g. be found in WO 95/07931 which is are hereby incorporated by reference.

A derivatized insulin peptide is thus human insulin or an insulin analogue which comprises at least one covalent modification such as a side-chain attached to one or more amino acids of the insulin peptide.

Herein, the naming of the derivatized insulin is done according to the following principles: The names are given as mutations and modifications (acylations) relative to human insulin. For the naming of the acyl moiety, the naming is done according to IUPAC nomenclature and in other cases as peptide nomenclature. For example, naming the acyl moiety:

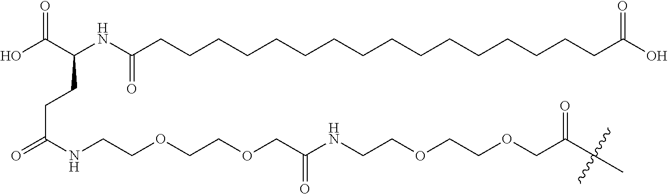

can be e.g. “octadecanedioyl-γ-L-Glu-OEG-OEG”, or “17-carboxyheptadecanoyl-γ-L-Glu-OEG-OEG”, wherein OEG is short hand notation for the amino acid —NH(CH₂)₂O(CH₂)₂OCH₂CO—, and γ-L-Glu (or g-L-Glu) is short hand notation for the L-form of the amino acid gamma glutamic acid moiety.

The acyl moiety of the modified peptides or proteins may be in the form of a pure enantiomer wherein the stereo configuration of the chiral amino acid moiety is either D or L (or if using the R/S terminology: either R or S) or it may be in the form of a mixture of enanti-omers (D and L/R and S). In one aspect of the invention the acyl moiety is in the form of a mixture of enantiomers. In one aspect the acyl moiety is in the form of a pure enantiomer. In one aspect the chiral amino acid moiety of the acyl moiety is in the L form. In one aspect the chiral amino acid moiety of the acyl moiety is in the D form.

In one aspect a derivatized insulin peptide according to the invention is an insulin peptide that is acylated in one or more amino acids of the insulin peptide.

In one aspect a derivatized insulin peptide according to the invention is soluble in propylene glycol. In another aspect a derivatized insulin peptide according to the invention is soluble in a propylene glycol solution comprising at least 20% w/w derivatized insulin peptide. In yet another aspect of the invention a derivatized insulin peptide according to the invention is soluble in a propylene glycol solution comprising at least 30% w/w derivatized insulin peptide.

In one aspect of the present invention, the derivatized insulin peptide is pH optimized before dissolution in the polar organic solvent to improve solubility in the polar organic solvent.

When using the term “pH optimized” it is herein meant that the derivatized insulin peptide has been dehydrated at a target pH which is at least 1 pH unit from the pI of the derivatized insulin peptide in aqueous solution. Thus, in one aspect of the invention, the target pH is more than 1 pH unit above the isoelectric point of the derivatized insulin peptide. In another aspect of the invention, the target pH is more than 1 pH unit below the isoelectric point of the derivatized insulin peptide. In a further aspect, the target pH is more than 1.5 pH units above or below the pI of the derivatized insulin peptide. In a yet further aspect, the target pH is 2.0 pH units or more above or below the pI of the derivatized insulin peptide. In a still further aspect, the target pH is 2.5 pH units or more above or below the pI of the derivatized insulin peptide. In yet a further aspect, the target pH is above the pI of the derivatized insulin peptide.

The term “dehydrated” as used herein in connection with a derivatized insulin peptide refers to a derivatized insulin peptide which has been dried from an aqueous solution. The term “target pH” as used herein refers to the aqueous pH which will establish when dehydrated derivatized insulin peptide is rehydrated in pure water to a concentration of approximately 40 mg/ml or more. The target pH will typically be identical to the pH of the aqueous derivatized insulin peptide solution from which the derivatized insulin peptide was recovered by drying. However, the pH of the derivatized insulin peptide solution will not be identical to the target pH, if the solution contains volatile acids or bases. It has been found that the pH history of the derivatized insulin peptide will be determinant for the amount of the derivatized insulin peptide, which may be solubilized in the polar organic solvent.

The term “the pI of the derivatized insulin peptide” as used herein refers to the isoelectric point of a derivatized insulin peptide.

The term “isoelectric point” as used herein means the pH value where the overall net charge of a macromolecule such as a peptide is zero. In peptides there may be several charged groups, and at the isoelectric point the sum of all these charges is zero. At a pH above the isoelectric point the overall net charge of the peptide will be negative, whereas at pH values below the isoelectric point the overall net charge of the peptide will be positive.

The pI of a protein may be determined experimentally by electrophoresis techniques such as electrofocusing:

A pH gradient is established in an anticonvective medium, such as a polyacrylamide gel. When a protein is introduced in to the system it will migrate under influence of an electric field applied across the gel. Positive charged proteins will migrate to the cathode. Eventually, the migrating protein reaches a point in the pH gradient where its net electrical charge is zero and is said to be focused. This is the isoelectric pH (pI) of the protein. The protein is then fixed on the gel and stained. The pI of the protein may then be determined by comparison of the position of the protein on the gel relative to marker molecules with known pI values.

The net charge of a protein at a given pH value may be estimated theoretically per a person skilled in the art by conventional methods. In essence, the net charge of protein is the equivalent to the sum of the fractional charges of the charged amino acids in the protein: aspartate (β-carboxyl group), glutamate (δ-carboxyl group), cysteine (thiol group), tyrosine (phenol group), histidine (imidazole side chains), lysine (ε-ammonium group) and arginine (guanidinium group). Additionally, one should also take into account charge of protein terminal groups (α-NH2 and α-COOH). The fractional charge of the ionisable groups may be calculated from the intrinsic pKa values.

The drying i.e. dehydration of the derivatized insulin peptide may be performed by any conventional drying method such e.g. by spray-, freeze-, vacuum-, open- and contact drying. In one aspect of the invention, the derivatized insulin peptide solution is dried to obtain a water content below about 10%. The water content may be below about 8%, below about 6%, below about 5%, below about 4%, below about 3%, below about 2% or below about 1% calculated on/measured by loss on drying test (gravimetric) as stated in the experimental part.

In one aspect of the invention the derivatized insulin peptide is spray dried. In a further aspect of the invention, the derivatized insulin peptide is freeze-dried.

In one aspect a derivatized insulin peptide according to the invention is an insulin peptide that is stabilised towards proteolytic degradation (by specific mutations) and further acylated at the B29-lysine. In another aspect a derivatized insulin peptide according to the invention is an insulin peptide that is an acylated, protease stabilized insulin, wherein the protease stabilised insulin analogue deviates from human insulin in one or more of the following deletions or substitutions: Q in position A18, A, G or Q in position A21, G or Q in position B1 or no amino acid residue in position B1, Q, S or T in position B3 or no amino acid residue in position B3, Q in position B13, no amino acid residue in position B27, D, E or R in position B28 and no amino acid in position B30.

In a broad aspect, a protease stabilised insulin is an insulin analogue wherein at least two hydrophobic amino acids have been substituted with hydrophilic amino acids relative to the parent insulin, wherein the substitutions are within or in close proximity to two or more protease cleavage sites of the parent insulin and wherein such insulin analogue optionally further comprises one or more additional mutations.

In another aspect, a protease stabilised insulin is an insulin analogue wherein
the amino acid in position A12 is Glu or Asp and/or the amino acid in position A13 is His, Asn, Glu or Asp and/or the amino acid in position A14 is Asn, Gln, Glu, Arg, Asp, Gly or His and/or the amino acid in position A15 is Glu or Asp; and
the amino acid in position B24 is His and/or the amino acid in position B25 is His and/or the amino acid in position B26 is His, Gly, Asp or Thr and/or the amino acid in position B27 is His, Glu, Gly or Arg and/or the amino acid in position B28 is His, Gly or Asp; and
   which optionally further comprises one or more additional mutations.

In another aspect, a protease stabilised insulin is an insulin analogue comprising an A-chain amino acid sequence of formula 1:

Formula (1)
(SEQ ID No: 1)
Xaa_{A(-2)}-Xaa_{A(-1)}-Xaa_{A0}-Gly-Ile-Val-Glu-Gln-Cys-
Cys-Xaa_{A8}-Ser-Ile-Cys-Xaa_{A12}-Xaa_{A13}-Xaa_{A14}-Xaa_{A15}-
Leu-Glu-Xaa_{A18}-Tyr-Cys-Xaa_{A21}

and a B-chain amino acid sequence of formula 2:

Formula (2)
(SEQ ID No: 2)
Xaa_{B(-2)}-Xaa_{B(-1)}-Xaa_{B0}-Xaa_{B1}-Xaa_{B2}-Xaa_{B3}-Xaa_{B4}-
His-Leu-Cys-Gly-Ser-Xaa_{B10}-Leu-Val-Glu-Ala-Leu-
Xaa_{B16}-Leu-Val-Cys-Gly-Glu-Arg-Gly-Xaa_{B24}-Xaa_{B25}-
Xaa_{B26}-Xaa_{B27}-Xaa_{B28}-Xaa_{B29}-Xaa_{B30}-Xaa_{B31}-Xaa_{B32}

wherein
Xaa_{A(-2)} is absent or Gly;
Xaa_{A(-1)} is absent or Pro;
Xaa_{A0} is absent or Pro;
Xaa_{A8} is independently selected from Thr and His;
Xaa_{A12} is independently selected from Ser, Asp and Glu;
Xaa_{A13} is independently selected from Leu, Thr, Asn, Asp, Gln, His, Lys, Gly, Arg, Pro, Ser and Glu;
Xaa_{A14} is independently selected from Tyr, Thr, Asn, Asp, Gln, His, Lys, Gly, Arg, Pro, Ser and Glu;
Xaa_{A15} is independently selected from Gln, Asp and Glu;
Xaa_{A18} is independently selected from Asn, Lys and Gln;
Xaa_{A21} is independently selected from Asn and Gln;
Xaa_{B(-2)} is absent or Gly;
Xaa_{B(-1)} is absent or Pro;
Xaa_{B0} is absent or Pro;
Xaa_{B1} is absent or independently selected from Phe and Glu;
Xaa_{B2} is absent or Val;
Xaa_{B3} is absent or independently selected from Asn and Gln;
Xaa_{B4} is independently selected from Gln and Glu;
Xaa_{B10} is independently selected from His, Asp, Pro and Glu;
Xaa_{B16} is independently selected from Tyr, Asp, Gln, His, Arg, and Glu;
Xaa_{B24} is independently selected from Phe and His;
Xaa_{B25} is independently selected from Asn, Phe and His;
Xaa_{B26} is absent or independently selected from Tyr, His, Thr, Gly and Asp;
Xaa_{B27} is absent or independently selected from Thr, Asn, Asp, Gln, His, Lys, Gly, Arg, Pro, Ser and Glu;
Xaa_{B28} is absent or independently selected from Pro, His, Gly and Asp;
Xaa_{B29} is absent or independently selected from Lys and Gln;
Xaa_{B30} is absent or Thr;
Xaa_{B31} is absent or Leu;
Xaa_{B32} is absent or Glu;
the C-terminal may optionally be derivatized as an amide;
   wherein the A-chain amino acid sequence and the B-chain amino acid sequence are connected by disulphide bridges between the cysteines in position 7 of the A-chain and the cysteine in position 7 of the B-chain, and between the cysteine in position 20 of the A-chain and the cysteine in position 19 of the B-chain and wherein the cysteines in position 6 and 11 of the A-chain are connected by a disulphide bridge.

With “desB30 insulin”, “desB30 human insulin” is meant insulin or an analogue thereof lacking the B30 amino acid residue.

By “parent insulin” is meant a naturally occurring insulin such as human insulin or porcine insulin. Alternatively, the parent insulin may be an insulin analogue.

In another aspect, a protease stabilised insulin is selected from the group consisting of the following compounds: A14E, B25H, desB30 human insulin; A14H, B25H, desB30 human insulin; A14E, B1E, B25H, desB30 human insulin; A14E, B16E, B25H, desB30 human insulin; A14E, B25H, B28D, desB30 human insulin; A14E, B25H, B27E, desB30 human insulin; A14E, B1E, B25H, B27E, desB30 human insulin; A14E, B1E, B16E, B25H, B27E, desB30 human insulin; A8H, A14E, B25H, desB30 human insulin; A8H, A14E, B25H, B27E, desB30 human insulin; A8H, A14E, B1E, B25H, desB30 human insulin; A8H, A14E, B1E, B25H, B27E, desB30 human insulin; A8H, A14E, B1E, B16E, B25H, B27E, desB30 human insulin; A8H, A14E, B16E, B25H, desB30 human insulin; A14E, B25H, B26D, desB30 human insulin; A14E, B1E, B27E, desB30 human insulin; A14E, B27E, desB30 human insulin; A14E, B28D, desB30 human insulin; A14E, B28E, desB30 human insulin; A14E, B1E, B28E, desB30 human insulin; A14E, B1E, B27E, B28E, desB30 human insulin; A14E, B1E, B25H, B28E, desB30 human insulin; A14E, B1E, B25H, B27E, B28E, desB30 human insulin; A14D, B25H, desB30 human insulin; B25N, B27E, desB30 human insulin; A8H, B25N, B27E, desB30 human insulin; A14E, B27E, B28E, desB30 human insulin; A14E, B25H, B28E, desB30 human insulin; B25H, B27E, desB30 human insulin; B1E, B25H, B27E, desb30 human insulin; A8H, B1E, B25H, B27E, desB30 human insulin; A8H, B25H, B27E, desB30 human insulin; B25N, B27D, desB30 human insulin; A8H, B25N, B27D, desB30 human insulin; B25H, B27D, desB309 human insulin; A8H, B25H, B27D, desB30 human insulin; A(−1)P, A(O)P, A14E, B25H, desB30 human insulin; A14E, B(−1)P, B(O)P, B25H, desB30 human insulin; A(−1)P, A(O)P, A14E, B(−1)P, B(O)P, B25H, desB30 human insulin; A14E, B25H, B30T, B31L, B32E human insulin; A14E, B25H human insulin; A14E, B16H, B25H, desB30 human insulin; A14E, B10P, B25H, desB30 human insulin; A14E, B10E, B25H, desB30 human insulin; A14E, B4E, B25H, desB30 human insulin; A14H, B16H, B25H, desB30 human insulin; A14H, B10E, B25H, desB30 human insulin; A13H, A14E, B10E, B25H, desB30 human insulin; A13H, A14E, B25H, desB30 human insulin; A14E, A18Q, B3Q, B25H, desB30 human insulin; A14E, B24H, B25H, desB30 human insulin; A14E, B25H, B26G, B27G, B28G, desB30 human insulin; A14E, A18Q, A21Q, B3Q, B25H, desB30 human insulin; A14E, A18Q, A21Q, B3Q, B25H, B27E, desB30 human insulin; A14E, A18Q, B3Q, B25H, desB30 human insulin; A13H, A14E, B1E, B25H, desB30 human insulin; A13N, A14E, B25H, desB30 human insulin; A13N, A14E, B1E, B25H, desB30 human insulin; A(−2)G, A(−1)P, A(O)P, A14E, B25H, desB30 human insulin; A14E, B(−2)G, B(−1)P, B(O)P, B25H, desB30 human insulin; A(−2)G, A(−1)P, A(O)P, A14E, B(−2)G, B(−1)P, B(O)P, B25H, desB30 human insulin; A14E, B27R, B28D, B29K, desB30 human insulin; A14E, B25H, B27R, B28D, B29K, desB30 human insulin; A14E, B25H, B26T, B27R, B28D, B29K, desB30 human insulin; A14E, B25H, B27R, desB30 human insulin; A14E, B25H, B27H, desB30 human insulin; A14E, A18Q, B3Q, B25H, desB30 human insulin; A13E, A14E, B25H, desB30 human insulin; A12E, A14E, B25H, desB30 human insulin; A15E, A14E, B25H, desB30 human insulin; A13E, B25H, desB30 human insulin; A12E, B25H, desB30 human insulin; A15E, B25H, desB30 human insulin; A14E, B25H, desB27, desB30 human insulin; A14E, B25H, B26D, B27E, desB30 human insulin; A14E, B25H, B27R, desB30 human insulin; A14E, B25H, B27N, desB30 human insulin; A14E, B25H, B27D, desB30 human insulin; A14E, B25H, B27Q, desB30 human insulin; A14E, B25H, B27E, desB30 human insulin; A14E, B25H, B27G, desB30 human insulin; A14E, B25H, B27H, desB30 human insulin; A14E, B25H, B27K, desB30 human insulin; A14E, B25H, B27P, desB30 human insulin; A14E, B25H, B27S, desB30 human insulin; A14E, B25H, B27T, desB30 human insulin; A13R, A14E, B25H, desB30 human insulin; A13N, A14E, B25H, desB30 human insulin; A13D, A14E, B25H, desB30 human insulin; A13Q, A14E, B25H, desB30 human insulin; A13E, A14E, B25H, desB30 human insulin; A13G, A14E, B25H, desB30 human insulin; A13H, A14E, B25H, desB30 human insulin; A13K, A14E, B25H, desB30 human insulin; A13P, A14E, B25H, desB30 human insulin; A13S, A14E, B25H, desB30 human insulin; A13T, A14E, B25H, desB30 human insulin; A14E, B16R, B25H, desB30 human insulin; A14E, B16D, B25H, desB30 human insulin; A14E, B16Q, B25H, desB30 human insulin; A14E, B16E, B25H, desB30 human insulin; A14E, B16H, B25H, desB30 human insulin; A14R, B25H, desB30 human insulin; A14N, B25H, desB30 human insulin; A14D, B25H, desB30 human insulin; A14Q, B25H, desB30 human insulin; A14E, B25H, desB30 human insulin; A14G, B25H, desB30 human insulin; A14H, B25H, desB30 human insulin; A8H, B10D, B25H human insulin; and A8H, A14E, B10E, B25H, desB30 human insulin.

Preferably, the acylated insulins of this invention are mono-substituted having only one acylation group attached to a lysine amino acid residue in the protease stabilised insulin molecule.

In one aspect, the acyl moiety attached to the protease stabilised insulin has the general formula:

Acy-AA1ₙ-AA2ₘ-AA3ₚ (I),

wherein n is 0 or an integer in the range from 1 to 3; m is 0 or an integer in the range from 1 to 10; p is 0 or an integer in the range from 1 to 10; Acy is a fatty acid or a fatty diacid comprising from about 8 to about 24 carbon atoms; AA1 is a neutral linear or cyclic amino acid residue; AA2 is an acidic amino acid residue; AA3 is a neutral, alkyleneglycol-containing amino acid residue; the order by which AA1, AA2 and AA3 appears in the formula can be interchanged independently; AA2 can occur several times along the formula (e.g., Acy-AA2-AA3₂-AA2-); AA2 can occur independently (=being different) several times along the formula (e.g., Acy-AA2-AA3₂-AA2-); the connections between Acy, AA1, AA2 and/or AA3 are amide (peptide) bonds which, formally, can be obtained by removal of a hydrogen atom or a hydroxyl group (water) from each of Acy, AA1, AA2 and AA3; and attachment to the protease stabilised insulin can be from the C-terminal end of a AA1, AA2, or AA3 residue in the acyl moiety of the formula (I) or from one of the side chain(s) of an AA2 residue present in the moiety of formula (I).

In another aspect, the acyl moiety attached to the protease stabilised insulin has the general formula Acy-AA1ₙ-AA2ₘ-AA3ₚ- (I), wherein AA1 is selected from Gly, D- or L-Ala, βAla, 4-aminobutyric acid, 5-aminovaleric acid, 6-aminohexanoic acid, D- or L-Glu-α-amide, D- or L-Glu-γ-amide, D- or L-Asp-α-amide, D- or L-Asp-β-amide, or a group of one of the formula:

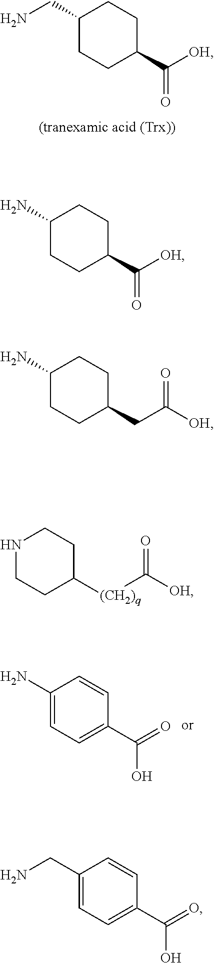

from which a hydrogen atom and/or a hydroxyl group has been removed and wherein q is 0, 1, 2, 3 or 4.

In another aspect, the acyl moiety attached to the protease stabilised insulin has the general formula Acy-AA1ₙ-AA2ₘ-AA3ₚ- (I), wherein AA1 is as defined above and AA2 is selected from L- or D-Glu, L- or D-Asp, L- or D-homoGlu or any of the following:

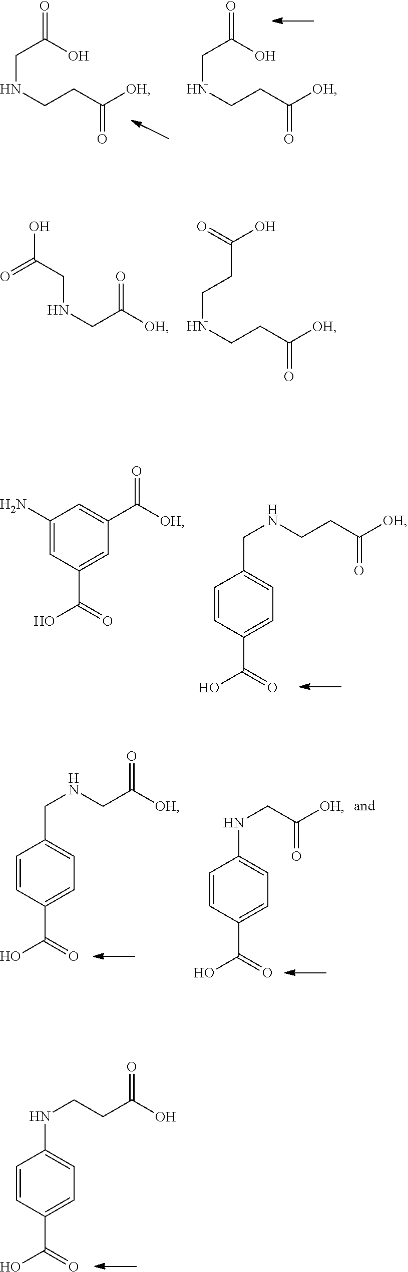

from which a hydrogen atom and/or a hydroxyl group has been removed and wherein the arrows indicate the attachment point to the amino group of AA1, AA2, AA3, or to the amino group of the protease stabilised insulin.

The neutral cyclic amino acid residue designated AA1 is an amino acid containing a saturated 6-membered carbocyclic ring, optionally containing a nitrogen hetero atom, and preferably the ring is a cyclohexane ring or a piperidine ring. Preferably, the molecular weight of this neutral cyclic amino acid is in the range from about 100 to about 200 Da.

The acidic amino acid residue designated AA2 is an amino acid with a molecular weight of up to about 200 Da comprising two carboxylic acid groups and one primary or secondary amino group.

The neutral, alkyleneglycol-containing amino acid residue designated AA3 is an alkylene-glycol moiety, optionally an oligo- or polyalkyleneglycol moiety containing a carboxylic acid functionality at one end and a amino group functionality at the other end.

Herein, the term alkyleneglycol moiety covers mono-alkyleneglycol moieties as well as oligo-alkyleneglycol moieties. Mono- and oligoalkyleneglycols comprises mono- and oligoethyl-eneglycol based, mono- and oligopropyleneglycol based and mono- and oligobutyleneglycol based chains, i.e., chains that are based on the repeating unit —CH₂CH₂O—, —CH₂CH₂CH₂O— or —CH₂CH₂CH₂CH₂O—. The alkyleneglycol moiety is monodisperse (with well defined length/molecular weight). Monoalkyleneglycol moieties comprise —OCH₂CH₂O—, —OCH₂CH₂CH₂O— or —OCH₂CH₂CH₂CH₂O— containing different groups at each end.

As mentioned herein, the order by which AA1, AA2 and AA3 appears in the acyl moiety with the formula (I) (Acy-AA1ₙ-AA2ₘ-AA3ₚ-) can be interchanged independently. Consequently, the formula Acy-AA1ₙ-AA2ₘ-AA3ₚ- also covers moieties like, e.g., the formula Acy-AA2ₘ-AA1ₙ-AA3₁- and the formula Acy-AA3ₚ-AA2ₘ-AA1ₙ-, wherein Acy, AA1, AA2, AA3, n, m and p are as defined herein.

As mentioned herein, the connections between the moieties Acy, AA1, AA2 and/or AA3 are formally obtained by amide bond (peptide bond) formation (—CONH—) by removal of water from the parent compounds from which they formally are build. This means that in order to get the complete formula for the acyl moiety with the formula (I) (Acy-AA1ₙ-AA2ₘ-AA3ₚ-, wherein Acy, AA1, AA2, AA3, n, m and p are as defined herein), one has, formally, to take the compounds given for the terms Acy, AA1, AA2 and AA3 and remove a hydrogen and/or hydroxyl from them and, formally, to connect the building blocks so obtained at the free ends so obtained.

Non-limiting, specific examples of the acyl moieties of the formula Acy-AA1ₙ-AA2ₘ-AA3ₚ-which may be present in the acylated insulin analogues of this invention are the following:

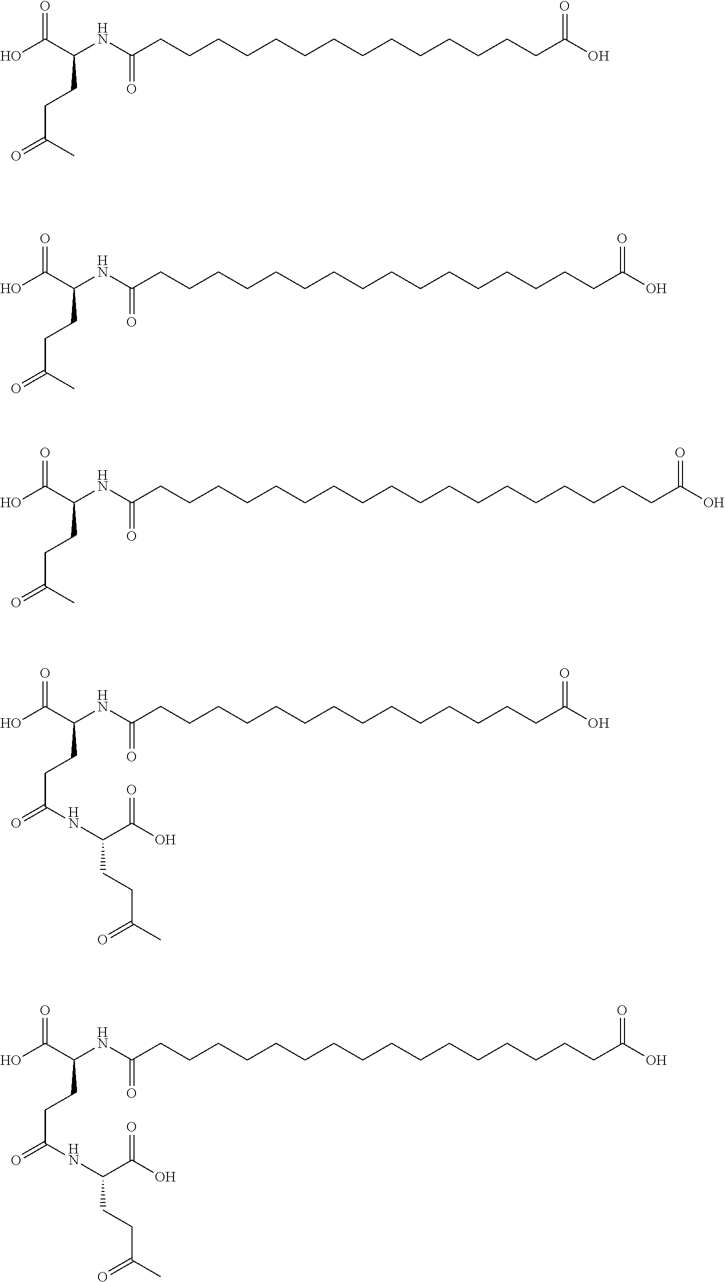

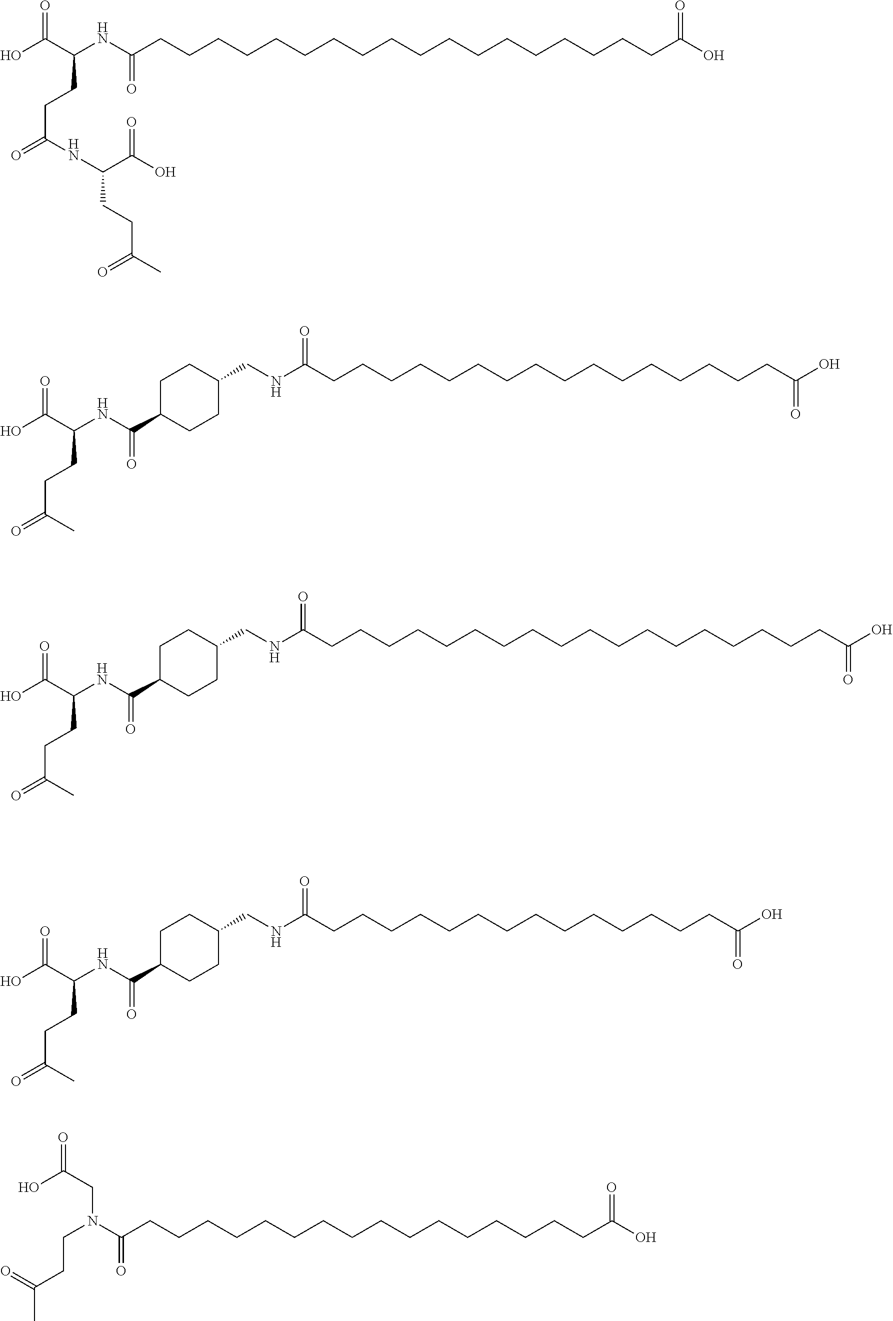

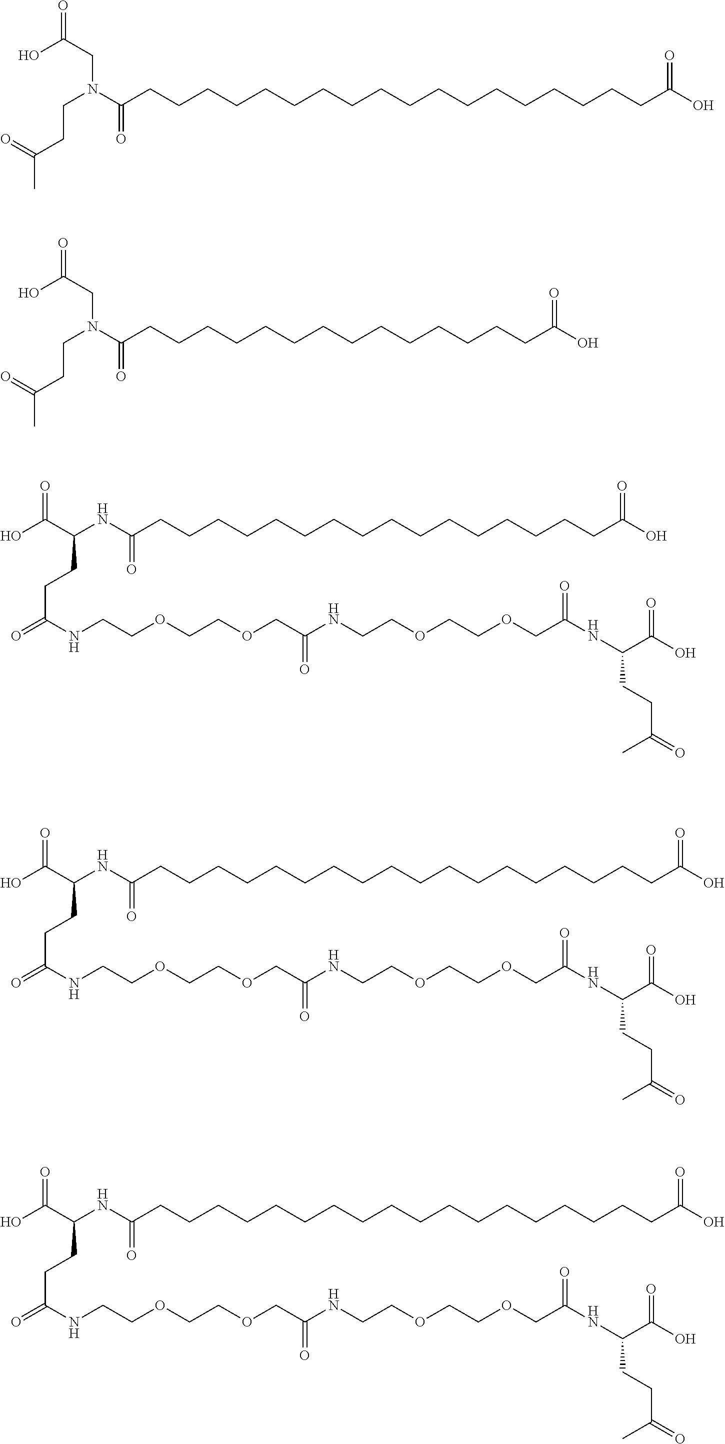

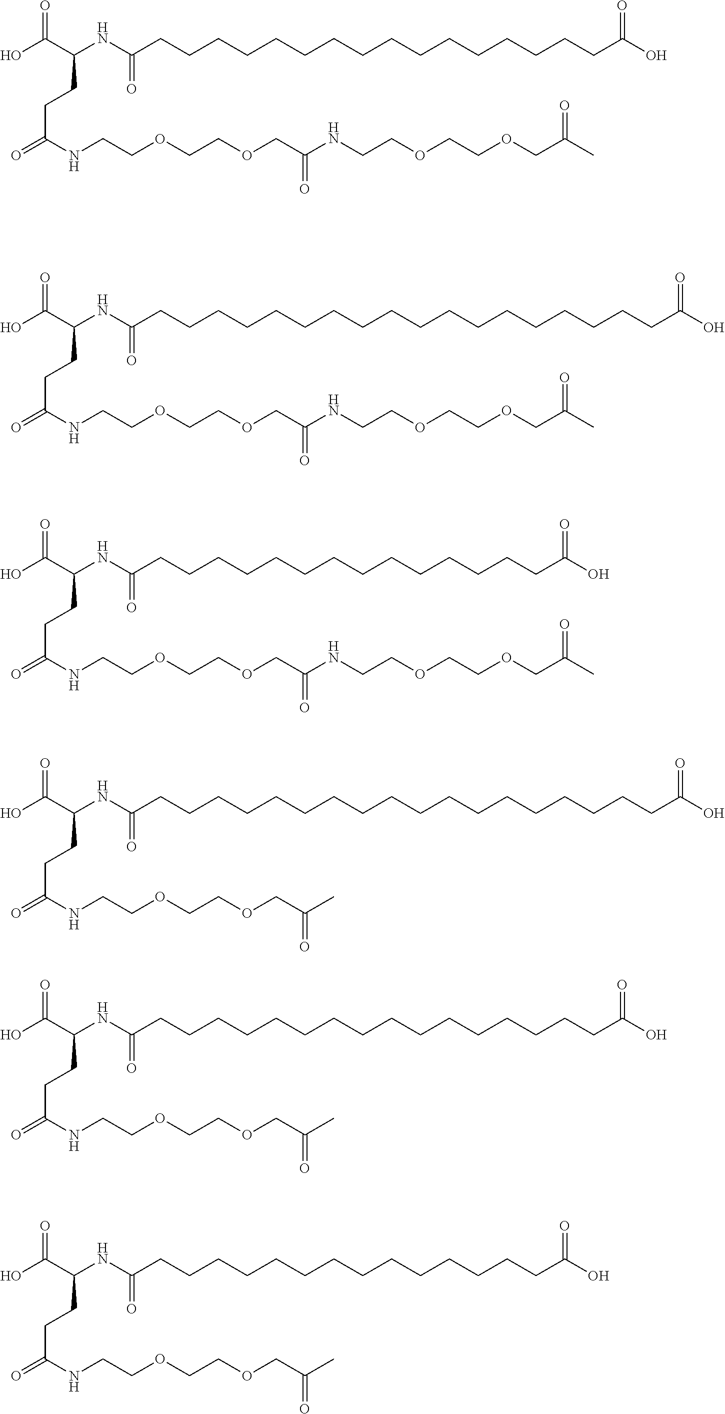

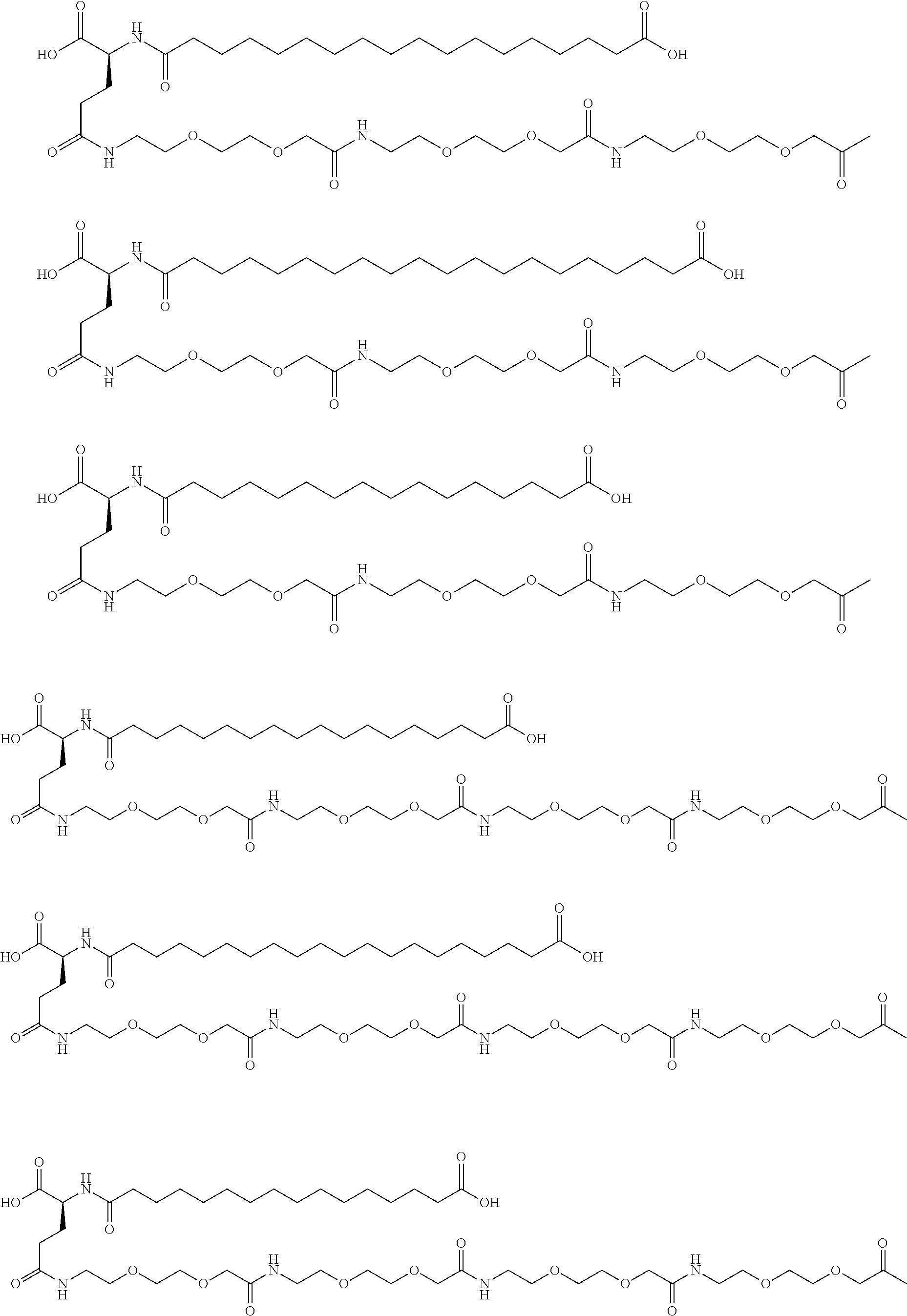

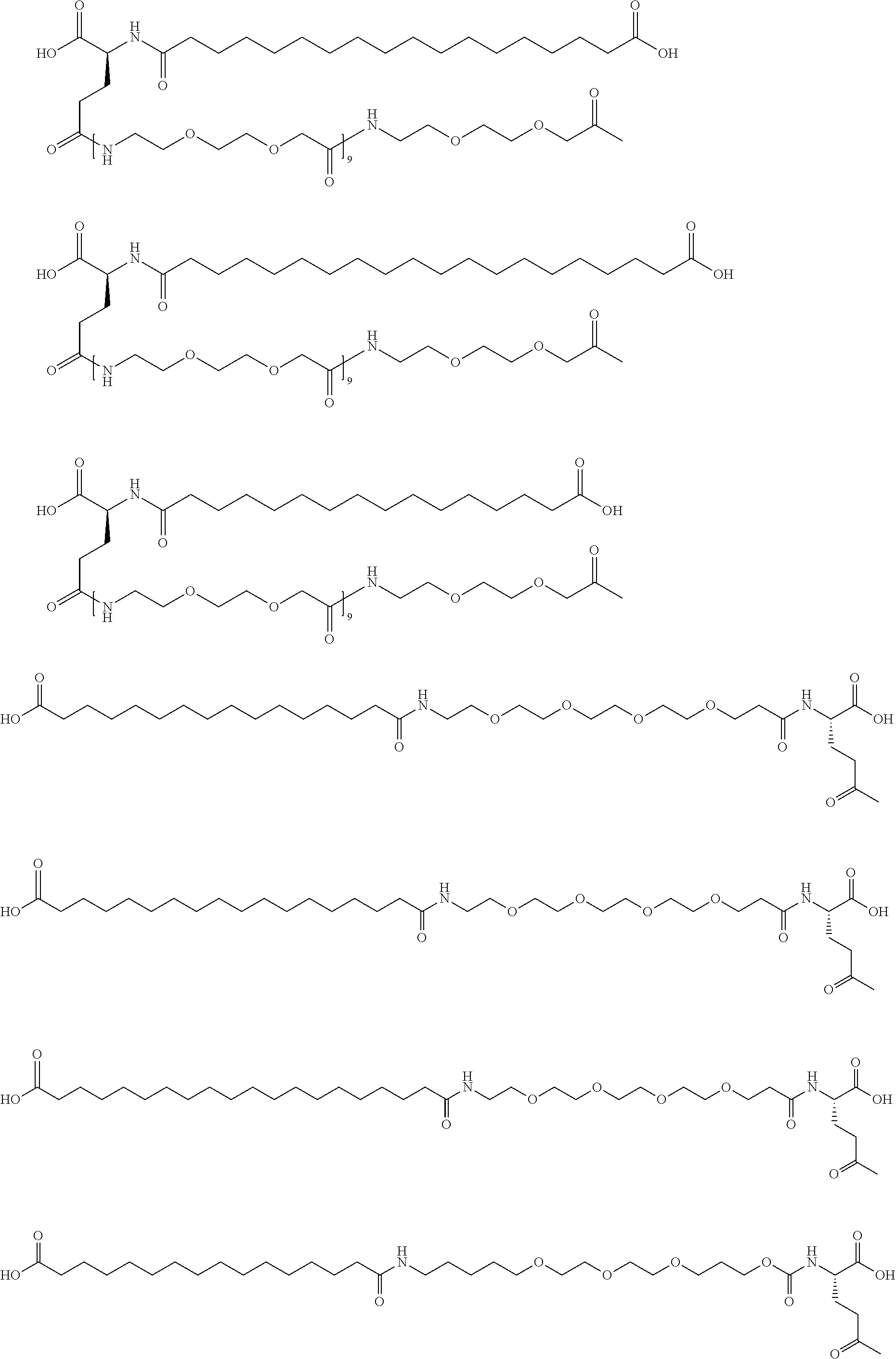

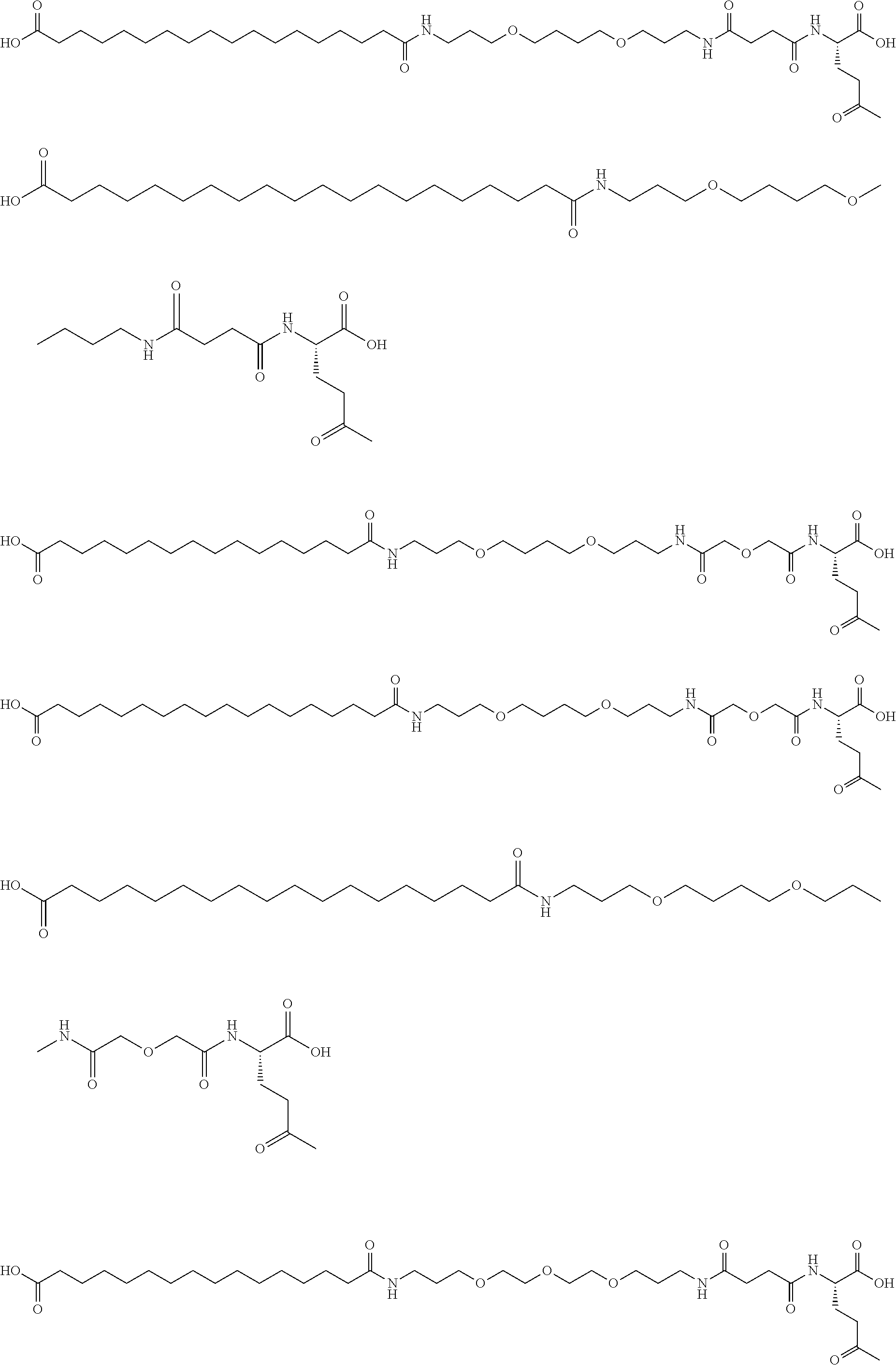

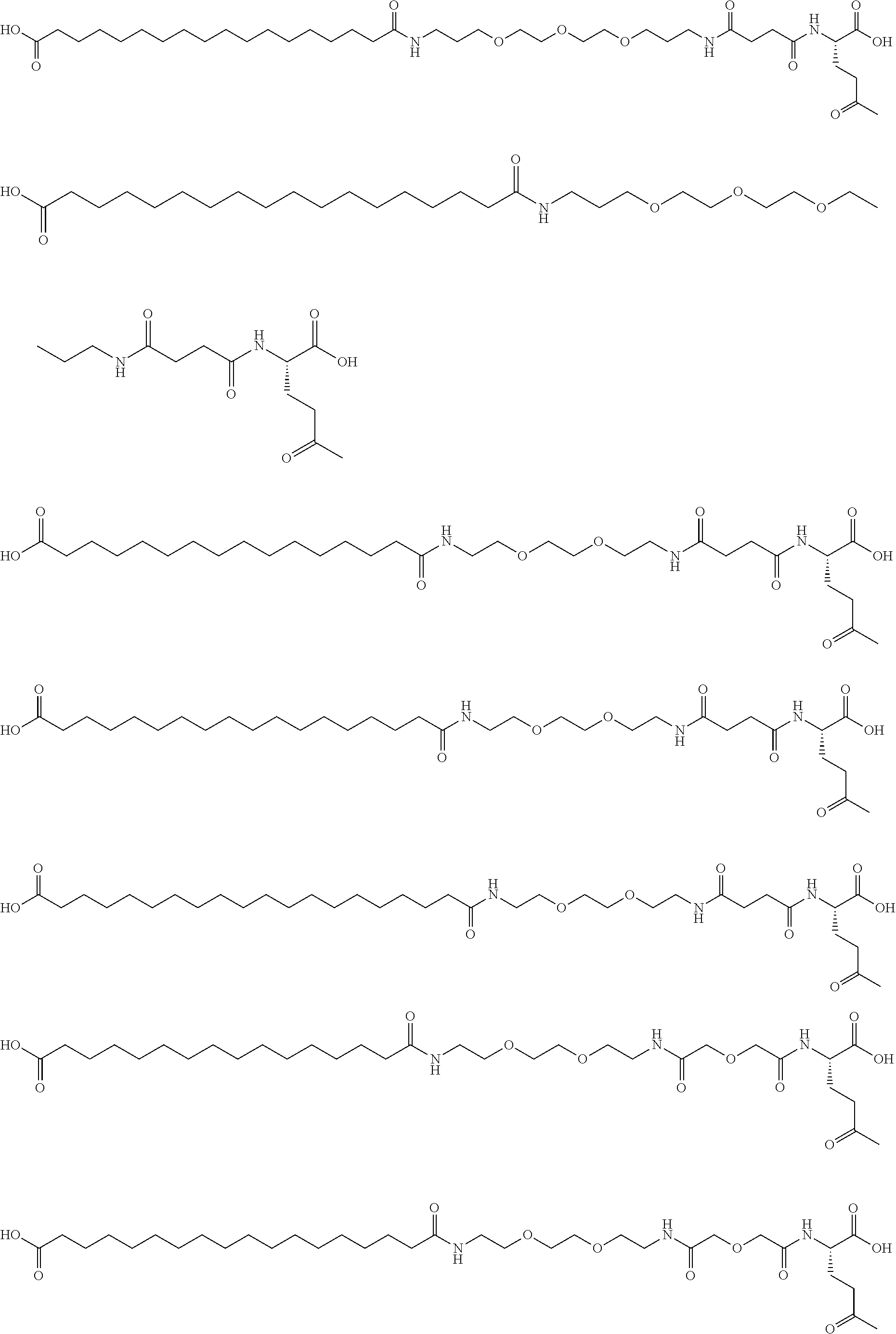

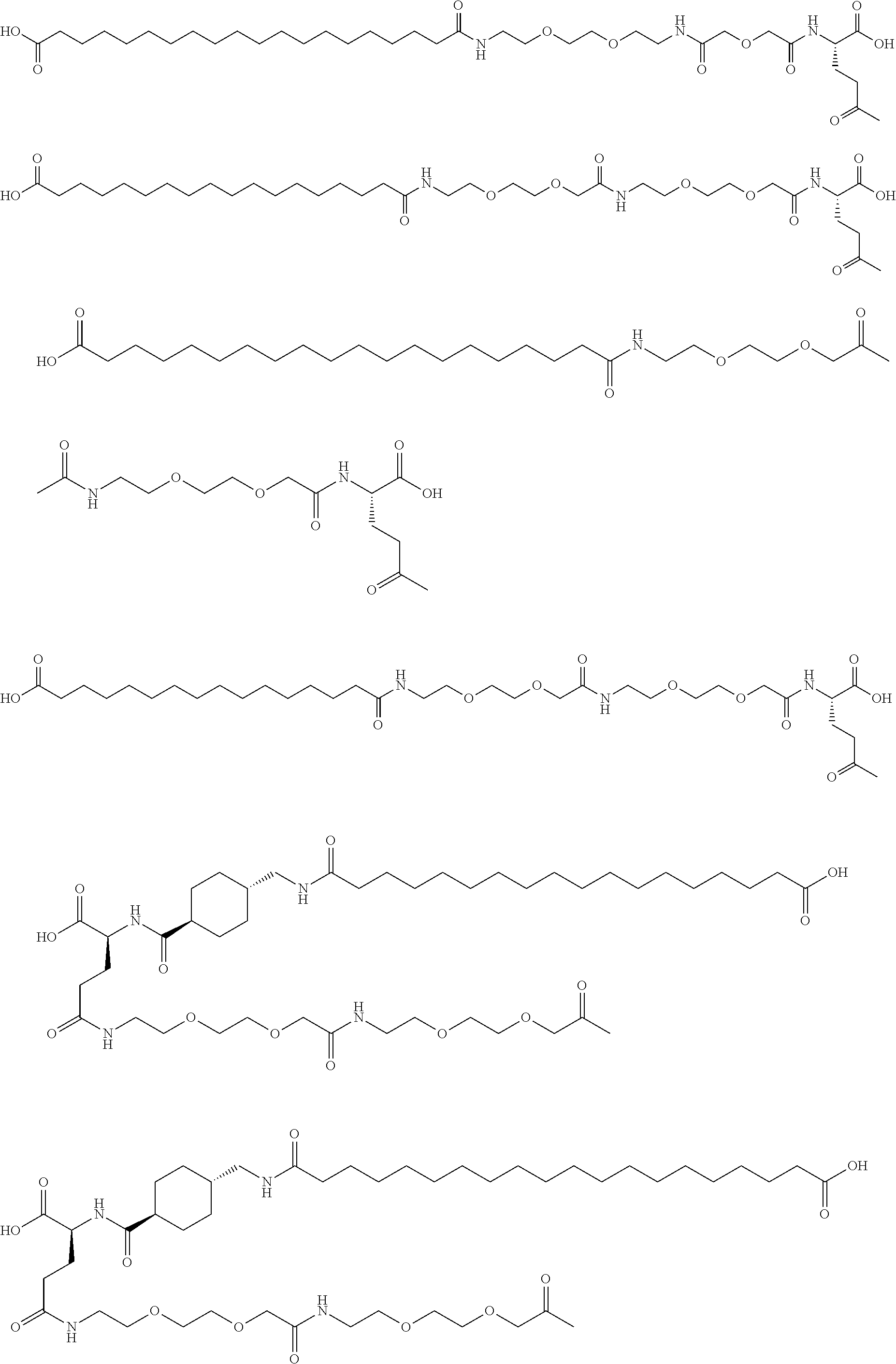

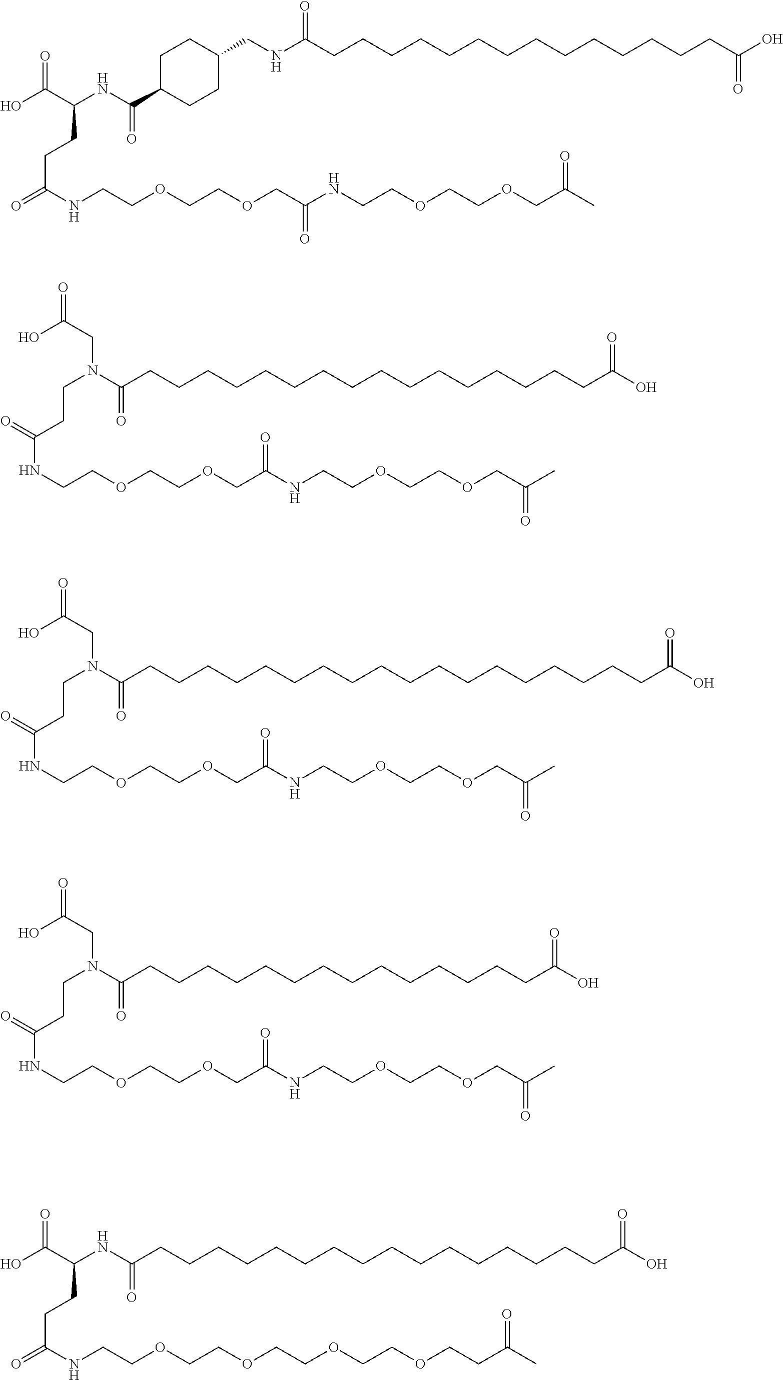

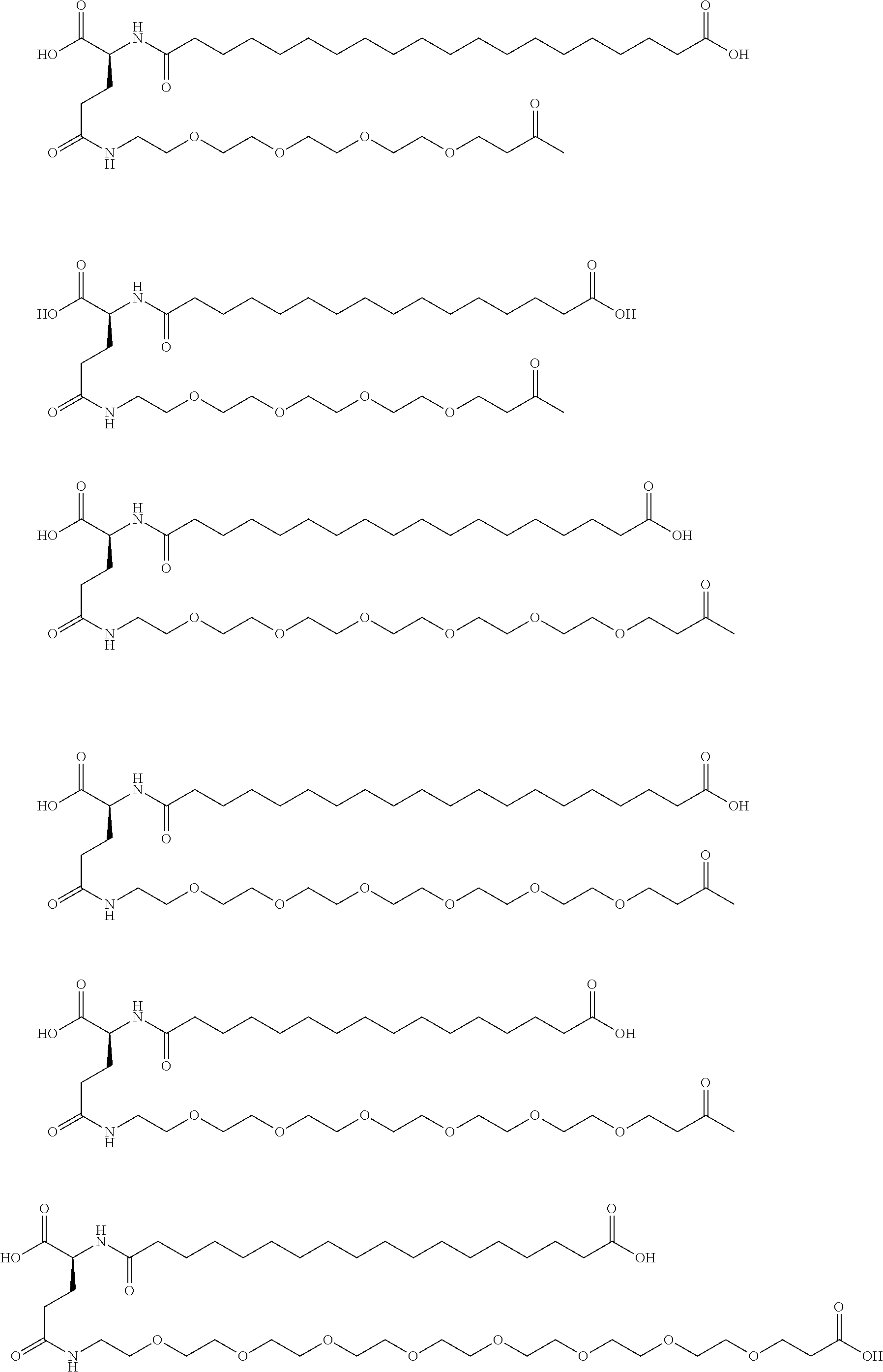

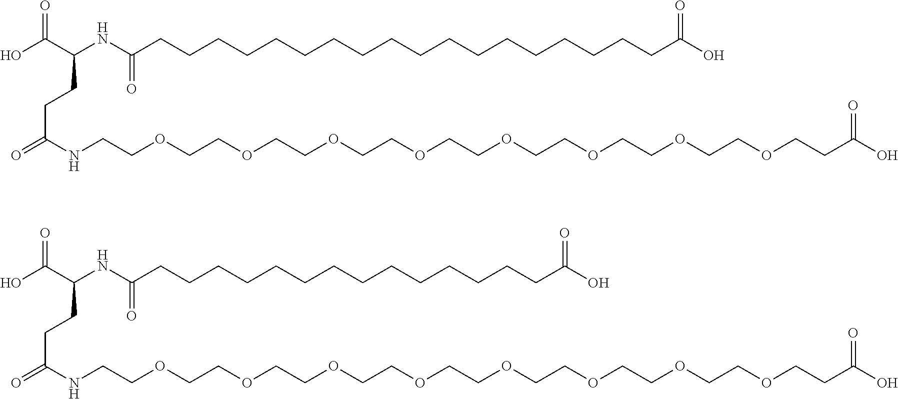

Any of the above non-limiting specific examples of acyl moieties of the formula Acy-AA1ₙ-AA2ₘ-AA3ₚ- can be attached to an epsilon amino group of a lysine residue present in any of the above non-limiting specific examples of insulin analogues thereby giving further specific examples of acylated insulin analogues of this invention.

The protease stabilized insulins can be converted into the acylated protease stabilized insulins of this invention by introducing the desired group of the formula Acy-AA1ₙ-AA2ₘ-AA3ₚ- in the lysine residue in the insulin analogue. The desired group of the formula Acy-AA1ₙ-AA2ₘ-AA3₁- can be introduced by any convenient method and many methods are disclosed in the prior art for such reactions. More details appear from the examples herein.

The present invention also relates to pharmaceutical compositions comprising acylated protease stabilized insulins wherein the C terminal amino acid residue in the A chain of the protease stabilized insulin is the A21 amino acid residue.

In a further aspect of the invention, the insulin derivative is selected from the group consisting of B29-N^{ε}-myristoyl-des(B30) human insulin, B29-N^{ε}-palmitoyl-des(B30) human insulin, B29-N^{ε}-myristoyl human insulin, B29-N^{ε}-palmitoyl human insulin, B28-N^{ε}-myristoyl Lys^{B28} Pro^{B29} human insulin, B28-N^{ε}-palmitoyl Lys^{B28} Pro^{B29} human insulin, B30-N^{ε}-myristoyl-Thr^{B29}Lys^{B39} human insulin, B30-N^{ε}-palmitoyl-Thr^{B29}Lys^{B39} human insulin, B29-N^{ε}-(N-palmitoyl-γ-glutamyl)-des(B30) human insulin, B29-N^{ε}-(N-lithocholyl-γ-glutamyl)-des(B30) human insulin, B29-N^{ε}-(ω-carboxyheptadecanoyl)-des(B30) human insulin and B29-N^{ε}-(ω-carboxyheptadecanoyl) human insulin.

In another aspect of the invention, the insulin derivative is B29-N(ε)-myristoyl-des(B30) human insulin.

In another aspect of the invention, the insulin derivative is B29K(N(ε)Octadecanedioyl-γGlu-OEG-OEG) A14E B25H desB30 human insulin.

In one aspect the water-free liquid pharmaceutical composition of the invention comprises a derivatized insulin peptide (a), at least one polar organic solvent (b) for the derivatized insulin peptide, at least one lipophilic component (c), and at least one surfactant (d), wherein the pharmaceutical composition is in the form of a clear solution, and wherein (b), (c) and (d) are in the relative amounts: 10-15% (b), 45-55% (c) and 30-40% (d).

In one aspect the water-free liquid pharmaceutical composition of the invention comprises a derivatized insulin peptide (a), at least one polar organic solvent (b) for the derivatized insulin peptide, at least one lipophilic component (c), and at least one surfactant (d), wherein the pharmaceutical composition is in the form of a clear solution, and wherein (b), (c) and (d) are in the relative amounts: 15% (b), 45% (c) and 40% (d).

In one aspect the water-free liquid pharmaceutical composition of the invention comprises a derivatized insulin peptide (a), a polar organic solvent (b) for the derivatized insulin peptide, a lipophilic component (c), and a surfactant (d), wherein the pharmaceutical composition is in the form of a clear solution, and wherein (b), (c) and (d) are in the relative amounts: 10-15% (b), 45-55% (c) and 30-40% (d), such as 15% (b), 45% (c) and 40% (d).

In one aspect the water-free liquid pharmaceutical composition of the invention comprises a derivatized insulin peptide (a), propylene glycol (b), glycerol monocaprylate (c), and labrasol (d), wherein the pharmaceutical composition is in the form of a clear solution, and wherein (b), (c) and (d) are in the relative amounts: 10-15% (b), 45-55% (c) and 30-40% (d), such as 15% (b), 45% (c) and 40% (d).

In one aspect the water-free liquid pharmaceutical composition of the invention comprises between 50 and 150 mg derivatized insulin peptide (a). In another aspect the water-free liquid pharmaceutical composition of the invention comprises between 70 and 130 mg derivatized insulin peptide (a). In yet another aspect the water-free liquid pharmaceutical composition of the invention comprises about 90 mg derivatized insulin peptide (a).

In one aspect the derivatized insulin peptide (a) is B29K(N(ε)Octadecanedioyl-γGlu-OEG-OEG) A14E B25H desB30 human insulin.

The production of polypeptides and peptides such as insulin is well known in the art. Polypeptides or peptides may for instance be produced by classical peptide synthesis, e.g. solid phase peptide synthesis using t-Boc or Fmoc chemistry or other well established techniques, see e.g. Greene and Wuts, “Protective Groups in Organic Synthesis”, John Wiley & Sons, 1999. The polypeptides or peptides may also be produced by a method which comprises culturing a host cell containing a DNA sequence encoding the (poly)peptide and capable of expressing the (poly)peptide in a suitable nutrient medium under conditions permitting the expression of the peptide. For (poly)peptides comprising non-natural amino acid residues, the recombinant cell should be modified such that the non-natural amino acids are incorporated into the (poly)peptide, for instance by use of tRNA mutants.

In one aspect a liquid or semisolid pharmaceutical composition according to the invention is shelf-stable.

The term “shelf-stable pharmaceutical composition” as used herein means a pharmaceutical composition which is stable for at least the period which is required by regulatory agencies in connection with therapeutic proteins. Preferably, a shelf-stable pharmaceutical composition is stable for at least one year at 5° C. Shelf-stability includes chemical stability as well as physical stability. Chemical instability involves degradation of covalent bonds, such as hydrolysis, racemization, oxidation or crosslinking. Chemical stability of the formulations is evaluated by means of reverse phase (RP-HPLC) and size exclusion chromatography SE-HPLC). In one aspect of the invention, the formation of peptide related impurities during shelf-life is less than 20% of the total peptide content. In a further aspect of the invention, the formation of peptide related during impurities during shelf-life is less than 10%. In a further aspect of the invention, the formation of peptide related during impurities during shelf-life is less than 5%. The RP-HPLC analysis is typically conducted in water-acetonitrile or water-ethanol mixtures. In one aspect, the solvent in the RP-HPLC step will comprise a salt such as Na₂SO₄, (NH₄)₂SO₄, NaCl, KCl, and buffer systems such as phosphate, and citrate and maleic acid. The required concentration of salt in the solvent may be from about 0.1 M to about 1 M, preferable between 0.2 M to 0.5 M, most preferable between 0.3 to 0.4 M. Increase of the concentration of salt requires an increase in the concentration of organic solvent in order to achieve elution from the column within a suitable time. Physical instability involves conformational changes relative to the native structure, which includes loss of higher order structure, aggregation, fibrillation, precipitation or adsorption to surfaces. Peptides such as insulin peptides, GLP-1 compounds and amylin compounds are known to be prone to instability due to fibrillation. Physical stability of the formulations may be evaluated by conventional means of e.g. visual inspection and nephelometry after storage of the formulation at different temperatures for various time periods. Conformational stability may be evaluated by circular dichroism and NMR as described by e.g. Hudson and Andersen, Peptide Science, vol 76 (4), pp. 298-308 (2004).

The biological activity of a derivatized insulin peptide may be measured in an assay as known by a person skilled in the art as e.g. described in WO 2005/012347.

In one aspect of the invention the pharmaceutical composition according to the invention is stable for more than 6 weeks of usage and for more than 3 years of storage.

In another aspect of the invention the pharmaceutical composition according to the invention is stable for more than 4 weeks of usage and for more than 3 years of storage.

In a further aspect of the invention the pharmaceutical composition according to the invention is stable for more than 4 weeks of usage and for more than two years of storage.

In an even further aspect of the invention the pharmaceutical composition according to the invention is stable for more than 2 weeks of usage and for more than two years of storage.

In an even further aspect of the invention the pharmaceutical composition according to the invention is stable for more than 1 weeks of usage and for more than one year of storage.

In one aspect, the pharmaceutical composition according to the invention is used for the preparation of a medicament for the treatment or prevention of hyperglycemia, type 2 diabetes, impaired glucose tolerance, and type 1 diabetes.

### FURTHER ASPECTS ACCORDING TO THE INVENTION

1. A water-free liquid or semisolid pharmaceutical composition comprising a derivatized insulin peptide (a), at least one polar organic solvent (b) for the derivatized insulin peptide, at least one lipophilic component (c), and optionally at least one surfactant (d) and/or at least one solid hydrophilic component (e)
2. A water-free liquid pharmaceutical composition comprising a derivatized insulin peptide (a), at least one polar organic solvent (b) for the derivatized insulin peptide, at least one lipophilic component (c), and optionally at least one surfactant (d), wherein the pharmaceutical composition is in the form of a clear solution.
3. The pharmaceutical composition according to aspect 1 or 2, which comprises at least one surfactant, wherein said pharmaceutical composition is spontaneously dispersible.
4. The pharmaceutical composition according to aspect 1 which comprises at least one solid hydrophilic component, wherein said pharmaceutical composition is in the form of an oily solution.
5. The pharmaceutical composition according to aspect 4 wherein said at least one hydrophilic component is at least one solid hydrophilic polymer.
6. The pharmaceutical composition according to any one of aspects 4 or 5, which is free of surfactant, wherein a surfactant has an HLB value which is at least 8.
7. The pharmaceutical composition according to any one of aspects 1-6, which comprises less than 10% w/w water.
8. The pharmaceutical composition according to any one of aspects 1-7, which comprises less than 5% w/w water.
9. The pharmaceutical composition according to any one of aspects 1-8, which comprises less than 2% w/w water.
10. The pharmaceutical composition according to any one of aspects 1-9, which comprises less than 1% w/w water.
11. The pharmaceutical composition according to any one of aspects 1-10, wherein the polar organic solvent is selected from the group consisting of polyols.
12. The pharmaceutical composition according to any one of aspects 1-11, wherein the polar organic solvent is selected from the group consisting of diols and triols.
13. The pharmaceutical composition according to any one of aspects 1-12, wherein the polar organic solvent is selected from the group consisting of propylene glycol, glycerol and mixtures thereof.
14. The pharmaceutical composition according to aspect 1-13, wherein the polar organic solvent is propylene glycol.
15. The pharmaceutical composition according to aspect 1-14, wherein the polar organic solvent is glycerol.
16. The pharmaceutical composition according to any one of aspects 1-15, wherein the derivatized insulin peptide is an acylated insulin or an acylated insulin analogue.
17. The pharmaceutical composition according to any one of aspects 1-15, wherein the derivatized insulin peptide is a protease stabilised insulin which has been derivatized in one or more positions.
18. The pharmaceutical composition according to any one of aspects 1-15, wherein the derivatized insulin peptide is a protease stabilised insulin which has been acylated in one or more positions.
19. The pharmaceutical composition according to any one of aspects 1-15, wherein the derivatized insulin peptide is a protease stabilised insulin which has been mono-substituted having only one acylation group attached to a lysine amino acid residue in the protease stabilised insulin molecule.
20. The pharmaceutical composition according to any one of aspects 1-15, wherein the derivatized insulin peptide is a protease stabilised insulin which has an acyl moiety attached to the protease stabilised insulin, wherein the acyl moiety has the general formula:

Acy-AA1ₙ-AA2ₘ-AA3ₚ- (I),

wherein n is 0 or an integer in the range from 1 to 3;

m is 0 or an integer in the range from 1 to 10;

p is 0 or an integer in the range from 1 to 10;

Acy is a fatty acid or a fatty diacid comprising from about 8 to about 24 carbon atoms;

AA1 is a neutral linear or cyclic amino acid residue;

AA2 is an acidic amino acid residue;

AA3 is a neutral, alkyleneglycol-containing amino acid residue;

and wherein the order by which AA1, AA2 and AA3 appears in the formula can be interchanged independently.
21. The pharmaceutical composition according to any one of aspects 1-20, wherein the derivatized insulin peptide is soluble in propylene glycol.
22. The pharmaceutical composition according to any one of aspects 1-21, wherein the derivatized insulin peptide is soluble in a propylene glycol solution comprising at least 20% w/w derivatized insulin peptide.
23. The pharmaceutical composition according to any one of aspects 1-22, wherein the derivatized insulin peptide is soluble in a propylene glycol solution comprising at least 30% w/w derivatized insulin peptide.
24. The pharmaceutical composition according to any one of the aspects 1-2 or 4-23, which does not comprise a surfactant, wherein a surfactant is defined as having an HLB value which is at least 8.
25. The pharmaceutical composition according to any one of the aspects 1-23 comprising a surfactant, wherein the surfactant is a non-ionic surfactant.
26. The pharmaceutical composition according to any one of the aspects 1-23 comprising a surfactant, wherein the surfactant is a polyoxyethylene containing surfactant.
27. The pharmaceutical composition according to any one of the aspects 1-23 comprising a surfactant, wherein the surfactant is caprylocaproyl macrogol-8 glycerides (such as Labrasol from Gattefosse.
28. The pharmaceutical composition according to any one of the aspects 1-23 comprising a surfactant, wherein the surfactant is a solid surfactant selected from the group consisting of a poloxamer and a mixture of poloxamers such as Pluronic F-127 or Pluronic F-68.
29. The pharmaceutical composition according to any one of the aspects 1-28, wherein the lipophilic component is mixable with propylene glycol.
30. The pharmaceutical composition according to any one of the aspects 1-28, wherein the lipophilic component is chosen such that a solution is obtained when the lipophilic component is mixed with propylene glycol.
31. The pharmaceutical composition according to any one of the aspects 1-28, wherein the lipophilic component is a phospholipid.
32. The pharmaceutical composition according to any one of the aspects 1-30, wherein the lipophilic component is a mono-, di- and/or tri-glyceride.
33. The pharmaceutical composition according to any one of the aspects 1-30 or 32, wherein the lipophilic component is a mono- and/or di-glyceride.
34. The pharmaceutical composition according to any one of the aspects 1-30, wherein the lipophilic component is propylene glycol caprylate.
35. The pharmaceutical composition according to any one of the aspects 1-30, wherein the lipophilic component is glycerol monocaprylate.
36. The pharmaceutical composition according to any one of the aspects 1-2, 4-24 or 29-35, which is liquid at room-temperature.
37. The pharmaceutical composition according to any one of the aspects 1-3, 6-23 or 25-34, which is semi-solid at room-temperature.
38. The pharmaceutical composition according to any one of the aspects 1-34, wherein (c) is liquid or semi-solid.
39. The pharmaceutical composition according to any one of the aspects 1-36, wherein (d) is liquid or semi-solid.
40. The pharmaceutical composition according to any one of the aspects 1-34, which comprises a solid hydrophilic component (e).
41. The pharmaceutical composition according to any one of the aspects 1-38 for use as a medicament in the treatment of hyperglycemia.
42. The pharmaceutical composition according to any one of the aspects 1-38 for use as a medicament.
43. The pharmaceutical composition according to any one of aspects 1-42, wherein the pharmaceutical composition is encapsulated in a hard or soft capsule.
44. The pharmaceutical composition according aspect 43, wherein the hard or soft capsule is enteric coated.
45. A method of producing a pharmaceutical composition according to any one of aspects 1-44.
46. A method of producing a pharmaceutical composition according to aspect 45 comprising the steps of:
(a) dissolving the derivatized insulin peptide in the polar organic solvent and
(b) subsequently mixing with the lipophilic component and optionally with the surfactant and/or hydrophilic component.
47. A method for treatment of hyperglycemia comprising oral administration of an effective amount of the pharmaceutical composition as defined in any of the aspects 1-38.
48. A method for treatment of obesity comprising oral administration of an effective amount of the pharmaceutical composition as defined in any of the aspects 1-38.
49. A method for treatment of binge eating or bulimia comprising oral administration of an effective amount of the pharmaceutical composition as defined in any of the aspects 1-38.

### EXAMPLES

The abbreviations used herein are standard abbreviations as e.g. the following: εAla is beta-alanyl, tBu is tert-butyl, γGlu is gamma L-glutamyl, OEG is [2-(2-aminoethoxy)ethoxy]ethylcarbonyl, RT is room temperature.
Insulin peptides were prepared using recombinant technology as known to the person skilled in the art. Derivatized insulin peptides were prepared as known to the person skilled in the art. As an exemplary preparation see Example 1.

### Example 1

### General Procedure of Preparation of Derivatized Insulin Peptides Such as A14E, B25H, B29K(N^{ε}-Hexadecanedioyl), desB30 Human Insulin

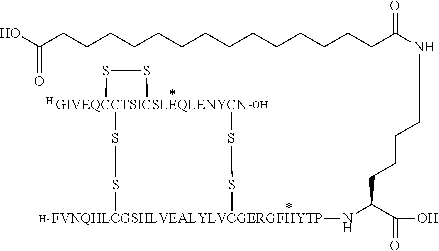

A14E, B25H, desB30 human insulin (500 mg) was dissolved in 100 mM aqueous Na₂CO₃ (5 mL), and pH adjusted to 10.5 with 1 N NaOH. Hexadecanedioic acid tert-butyl ester N-hydroxysuccinimide ester was dissolved in acetonitrile (10 W/V %) and added to the insulin solution and heated gently under warm tap, to avoid precipitation and left at room temperature for 30 minutes. The mixture was lyophilised. The solid was dissolved in ice-cold 95% trifluoroacetic acid (containing 5% water) and kept on ice for 30 minutes. The mixture was concentrated in vacuo and re-evaporated from dichloromethane. The residue was dissolved in water, and pH was adjusted to neutral (6-7) and the mixture was lyophilised. The resulting insulin was purified by ion exchance chromatography on a Source 15Q 21 ml column, several runs, eluting with a gradient of 15 to 300 mM ammonium acetate in 15 mM Tris, 50v/v % ethanol, pH 7.5 (acetic acid). Final desalting of pure fractions were performed on a RPC 3 mL column eluting isocraticlly with 0.1v/v % TFA, 50 v/v % ethanol. The resulting pure insulin was lyophilised.

LC-MS (electrospray): m/z=1483.2 (M+4)/4. Calcd: 1483.5

### Example 2

### Oral Administration of the Derivatized Insulin Peptide B29K(N^{ε}Octadecanedioyl-γGlu-OEG-OEG) A14E B25H desB30 Human Insulin

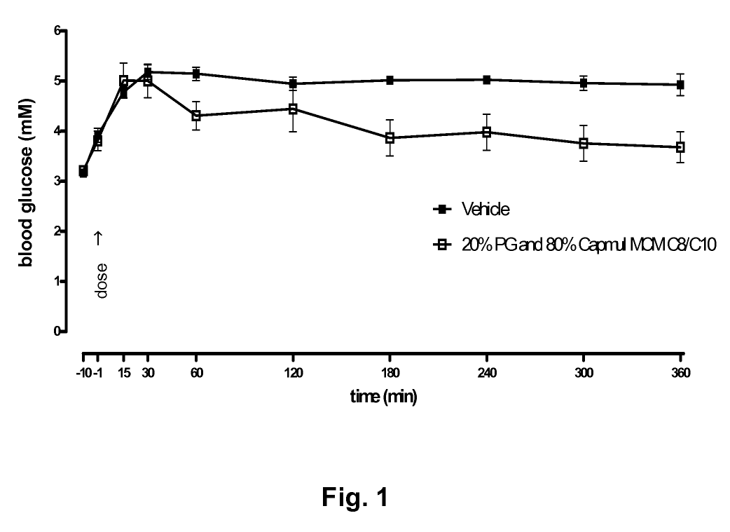

Lyophilized pH neutral powder of insulin derivative B29K(N^{ε}-Octadecanedioyl-γGlu-OEG-OEG) A14E B25H desB30 human insulin (4 ml/kg) of 800 nmol/kg) was dissolved in propylene glycol at RT and mixed after complete dissolution with Capmul MCM C8/10 at RT by magnetic stirring to result in a clear homogenous liquids.
The obtained lipophilic component based pharmaceutical composition had 20% propylene glycol and 80% Capmul MCM C8/10. The pharmaceutical composition was administered to overnight fasted male Wistar rats (mean±SEM, n=6). A vehicle without insulin derivative was administrated as control. The results are shown in FIG. 1.

### Example 3

### Plasma Exposure of the Derivatized Insulin Peptide B29K(N^{ε}-Octadecanedioyl-γGlu-OEG-OEG) A14E B25H desB30 Human Insulin Formulated with Misc. Lipophilic Components

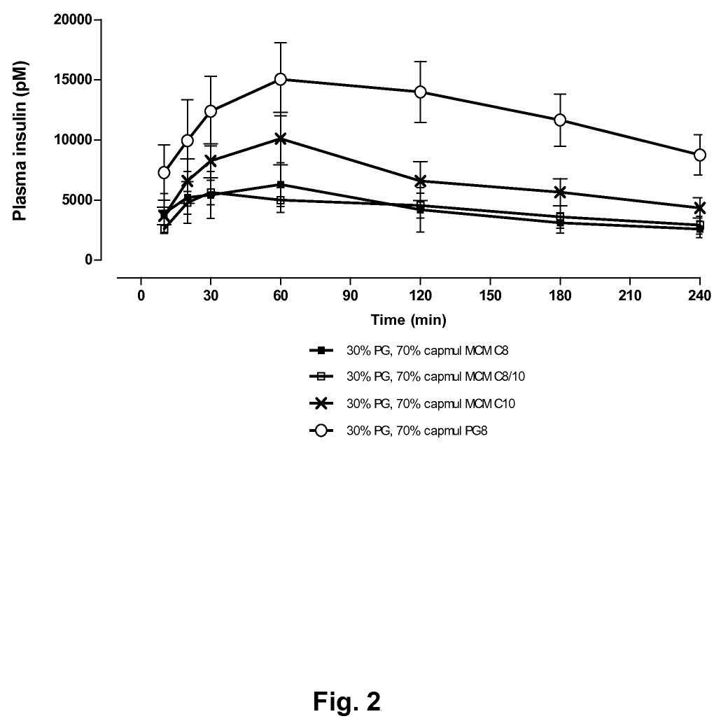

Lyophilized pH neutral powder of the insulin derivative B29K(N^{ε}-Octadecanedioyl-γGlu-OEG-OEG) A14E B25H desB30 human insulin was dissolved in propylene glycol at RT and after complete dissolution, the according lipid component was added and mixed by magnetic stirring at RT for 5 to 10 minutes to result in clear to slightly opaque liquids.
Plasma exposure (in pM) of the insulin derivative B29K(N^{ε}-Octadecanedioyl-γGlu-OEG-OEG) A14E B25H desB30 human insulin was measured after intestinal injection of 60 nmol/kg (0.4 ml/kg) into the mid-jejunum of fasted male SPRD rats (mean±sem, n=5-6) formulated in the following lipophilic component based delivery systems:
1) 30% propylene glycol and 70% capmul mcm c8,
2) 30% propylene glycol and 70% capmul mcm c8/10,
3) 30% propylene glycol and 70% capmul mcm c10,
4) 30% propylene glycol and 70% capmul pg8.
The delivery system with the insulin derivative dissolved in 30% propylene glycol and 70% propylene glycol caprylate (capmul pg8) showed highest plasma exposure.
The results are shown in FIG. 2.

### Example 4

### Plasma Exposure of the Derivatized Insulin Peptide B29K(N^{ε}-Octadecanedioyl-γGlu-OEG-OEG) A14E B25H desB30 Human Insulin Formulated with or without Surfactant

Lyophilized pH neutral powder of the insulin derivative B29K(N^{ε}-Octadecanedioyl-γGlu-OEG-OEG) A14E B25H desB30 human insulin was dissolved in propylene glycol at RT and after complete dissolution, the according lipid component and the according surfactant were added and mixed by magnetic stirring at RT for 5 to 10 minutes to result in clear homogenous liquids.

Plasma exposure (in pM) of the insulin derivative B29K(N^{ε}-Octadecanedioyl-γGlu-OEG-OEG) A14E B25H desB30 human insulin was measured after intestinal injection of 60 nmol/kg (0.4 ml/kg) into the mid-jejunum of fasted male SPRD rats (mean±SEM, n=5-6) formulated in the following pharmaceutical compositions:

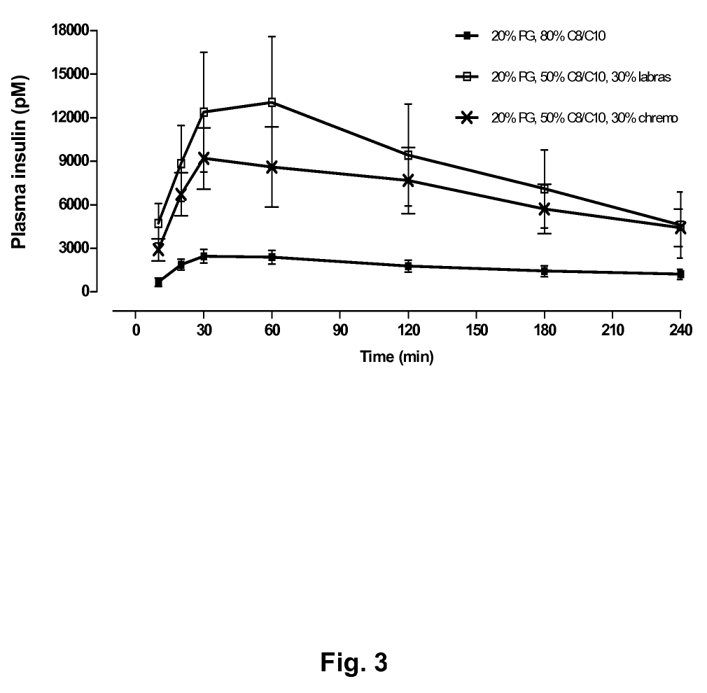

20% propylene glycol and 80% Capmul MCM C8/C10,
20% propylene glycol, 50% Capmul MCM C8/10 and 30% Labrasol,
20% propylene glycol, 50% Capmul MCM C8/C10 and 30% Chremophor RH40.
The results are shown in FIG. 3.

### Example 5

### Blood Glucose Lowering Effect of a Derivatized Insulin Peptide vs. a Non-Derivatized Insulin Peptide Formulated in a SEDDS

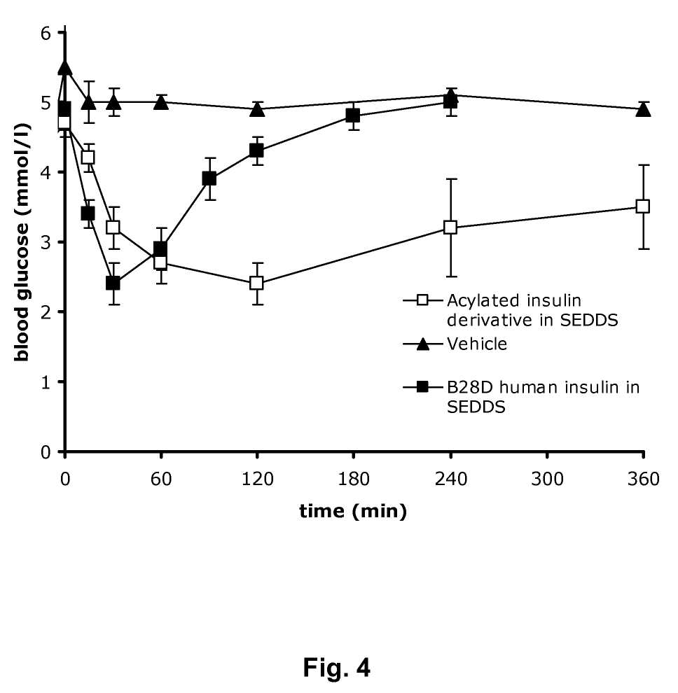

Lyophilized pH neutral powder of the according insulin was dissolved in propylene glycol at RT and after complete dissolution, the lipophilic component and the surfactant (melted together at 58C) were added and mixed by magnetic stirring at 35C for 5 to 10 minutes to result in clear homogenous liquids but solidified at RT. The samples where heated up to body temperature to become liquid before oral administration. The resulting SEDDS compositions consisted of the according insulin dissolved in 62.5% propylene glycol, 31.25% Capmul MCM 10 and 6.25% poloxamer 407 (mean±SEM, n=6). Blood glucose lowering effect was measured after oral administration (4 ml/kg) of 4800 nmol/kg of the derivatized insulin peptide B29(N^{ε}-hexadecandioyl-γ-L-Glu) A14E B25H desB30 human insulin in a SEDDS or 4800 nmol/kg B28D human insulin in SEDDS to overnight fasted male SPRD rats. A vehicle without insulin was administrated as control. The derivatized insulin peptide in the SEDDS pharmaceutical composition showed sustained blood glucose lowering effect in comparison with non-derivatized insulin.
The results are shown in FIG. 4.

### Example 6

### Method of Injection Intraintestinally (Jejunum) Rat for PK Studies

Anaesthetized rats were dosed intraintestinally (into jejunum) with the (derivatized) insulin peptide. Plasma concentrations of the employed compounds as well as changes in blood glucose were measured at specified intervals for 4 hours post-dosing. Pharmacokinetic parameters were subsequently calculated using WinNonLin.
Male Sprague-Dawley rats (Taconic), weighing 250-300 g, fasted for ˜18 h were anesthetized.
The anesthetized rat was placed on a homeothermic blanket stabilized at 37° C. A 20 cm polyethylene catheter mounted a 1-ml syringe was filled with insulin formulation or vehicle. A 4-5 cm midline incision was made in the abdominal wall. The catheter was gently inserted into mid-jejunum ˜50 cm from the caecum by penetration of the intestinal wall. If intestinal content was present, the application site was moved ±10 cm. The catheter tip was placed approx. 2 cm inside the lumen of the intestinal segment and fixed without the use of ligatures. The intestines were carefully replaced in the abdominal cavity and the abdominal wall and skin were closed with autoclips in each layer. At time 0, the rats were dosed via the catheter, 0.4 ml/kg of test compound or vehicle.
Blood samples for the determination of whole blood glucose concentrations were collected in heparinised 10 μl capillary tubes by puncture of the capillary vessels in the tail tip. Blood glucose concentrations were measured after dilution in 500 μl analysis buffer by the glucose oxidase method using a Biosen autoanalyzer (EKF Diagnostic Gmbh, Germany). Mean blood glucose concentration courses (mean±SEM) were made for each compound.
Samples were collected for determination of the plasma insulin peptide concentration. 100 μl blood samples were drawn into chilled tubes containing EDTA. The samples were kept on ice until centrifuged (7000 rpm, 4° C., 5 min), plasma was pipetted into Micronic tubes and then frozen at 20° C. until assay. Plasma concentrations of the insulin analogs were measured using a LOCI assay.
Blood samples were drawn at t=−10 (for blood glucose only), at t=−1 (just before dosing) and at specified intervals for 4 hours post-dosing.
Plasma concentration-time profiles were analysed by a non-compartmental pharmacokinetics analysis using WinNonlin Professional (Pharsight Inc., Mountain View, Calif., USA).
Calculations were performed using individual concentration-time values from each animal.

### Example 7

### Plasma Exposure of Derivatized Insulin Peptides Formulated in SEDDS

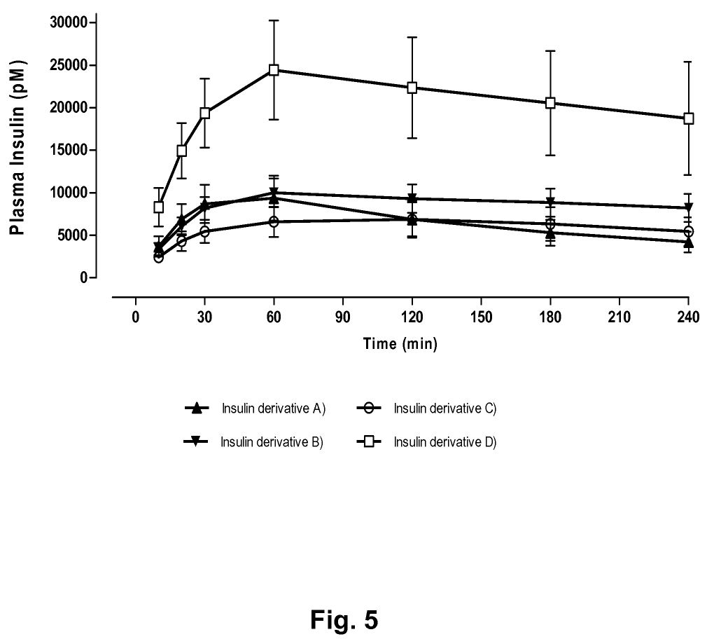

Samples of insulin derivatives: A) A14E, B25H, B29K (N(eps)Octadecanedioyl-gGlu-OEG-OEG), desB30 human insulin, insulin derivative, B) A14E, B16H, B25H, B29K ((N(eps)Eicosanedioyl-gGlu-[2-(2-{2-[2-(2-aminoethoxy)ethoxy]acetylamino}ethoxy)ethoxy]acetyl)), desB30 human insulin, insulin derivative, C) A14E, B25H, B29K (N(eps) [2-(2-{2-[2-(2-{2-[(S)-4-carboxy-4-(19-carboxynonadecanoylamino)butyrylamino]ethoxy}ethoxy) acetylamino]ethoxy}ethoxy)acetyl]), desB30 human insulin and insulin derivative and D) A14E, B16H, B25H, B29K(N(eps)-[2-(2-[2-(2-[2-(Octadecandioyl-gGlu)amino]ethoxy)ethoxy]acetylamino)ethoxy]ethoxy)acetyl]), desB30 human insulin were prepared and consisted of:
(120 nmol/kg) Insulin derivative A), B), C) or D) formulated in 15% propylene glycol, 55% Capmul MCM and 30% Labrasol.
The samples were prepared by the following method:
Lyophilized pH neutral powder of the according insulin derivative was dissolved in propylene glycol at RT and after complete dissolution, the according lipid component and the according surfactant were added and mixed by magnetic stirring at RT for 5 to 10 minutes to result in clear homogenous liquids.
Plasma exposure (in pM) of the insulin derivatives was determined after intestinal injection of 120 nmol/kg (0.4 ml/kg) into the mid-jejunum of fasted male SPRD rats (mean±SEM, n=6). The results are shown in FIG. 5.

### Example 8

### Plasma Exposure of Derivatized Insulin Peptides Formulated in SEDDS

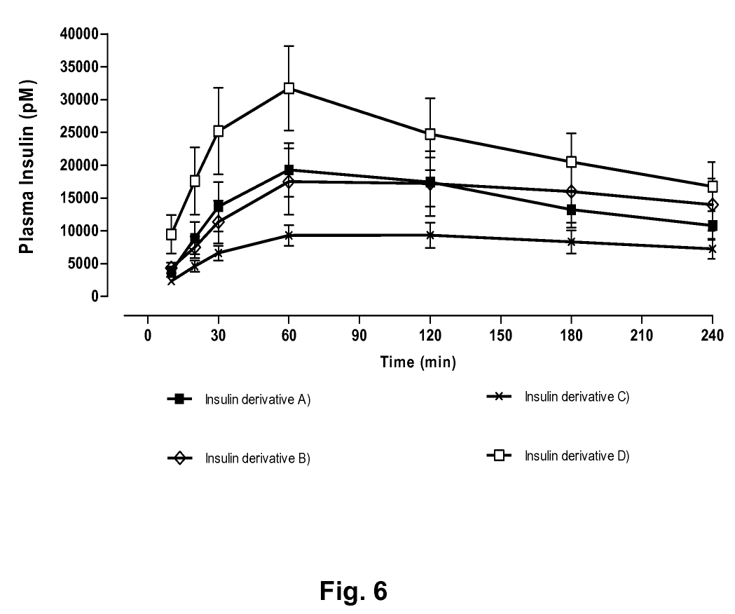

Samples of insulin derivatives: A) A14E, B25H, B29K (N(eps)Octadecanedioyl-gGlu-OEG-OEG), desB30 human insulin, insulin derivative, B) A14E, B16H, B25H, B29K ((N(eps)Eicosanedioyl-gGlu-[2-(2-{2-[2-(2-aminoethoxy)ethoxy]acetylamino}ethoxy)ethoxy]acetyl)), desB30 human insulin, insulin derivative, C) A14E, B25H, B29K (N(eps) [2-(2-{2-[2-(2-{2-[(S)-4-carboxy-4-(19-carboxynonadecanoylamino)butyrylamino]ethoxy}ethoxy)acetylamino]ethoxy}ethoxy)acetyl]), desB30 human insulin and insulin derivative, and D) A14E, B16H, B25H, B29K(N(eps)-[2-(2-[2-(2-[2-(Octadecandioyl-gGlu)amino]ethoxy)ethoxy]acetylamino)ethoxy]ethoxy)acetyl]), desB30 human insulin were prepared and consisted of:
(120 nmol/kg) Insulin derivative A), B), C) or D) formulated in 55% propylene glycol, 35% Capmul MCM and 10% Poloxamer 407.
The samples were prepared by the following method:
Lyophilized pH neutral powder of the according insulin derivative was dissolved in propylene glycol at RT and after complete dissolution, the according lipid component and the according surfactant were added and mixed by magnetic stirring at RT for 5 to 10 minutes to result in clear homogenous liquids.
Plasma exposure (in pM) of the insulin derivatives was determined after intestinal injection of 120 nmol/kg (0.4 ml/kg) into the mid-jejunum of fasted male SPRD rats (mean±SEM, n=6). The results are shown in FIG. 6.

### Example 9

### Plasma Exposure of Derivatized Insulin Peptides Formulated in SEDDS

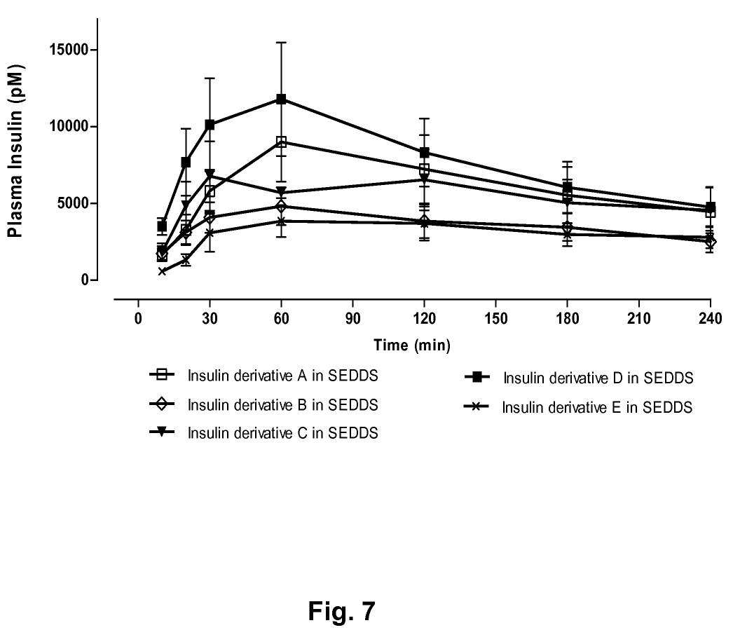

Samples of insulin derivatives: A) A14E, B25H, B29K (N(eps)Octadecanedioyl-gGlu-OEG-OEG), desB30 human insulin, insulin derivative, B) A1N-octadecandioyl-gamma-L-glutamyl-[2-(2-{2-[2-(2-aminoethoxy)ethoxy]acetylamino}ethoxy)ethoxy]acetyl A14E B25H □B29R desB30 human insulin, insulin derivative, C) A14E, B25H, B29K(N(eps)Octadecandioyl-g-Glu), desB30 human Insulin, insulin derivative, D) A14E, B25H, (N(eps)-[2-(2-[2-(2-[2-(Octadecandioyl-gGlu)amino]ethoxy)ethoxy]acetylamino)ethoxy]ethoxy)acetyl]), desB27, desB30 human insulin and insulin derivative, and E) A14E, B25H, B29K(N(eps)lcosandioyl-gGlu), desB30 human insulin were prepared and consisted of:
Insulin derivative A), B), C), D) or E) formulated in 55% propylene glycol, 35% Capmul MCM and 10% Poloxamer 407.
The samples were prepared by the following method:
Lyophilized pH neutral powder of the according insulin derivative was dissolved in propylene glycol at RT and after complete dissolution, the according lipid component and the according surfactant were added and mixed by magnetic stirring at RT for 5 to 10 minutes to result in clear homogenous liquids.
Plasma exposure (in pM) of the insulin derivatives was determined after intestinal injection of 120 nmol/kg (0.4 ml/kg) into the mid-jejunum of fasted male SPRD rats (mean±SEM, n=6). The results are shown in FIG. 7.

### Example 10

### Plasma Exposure of Derivatized Insulin Peptide Dissolved in Water or Propylene Glycol

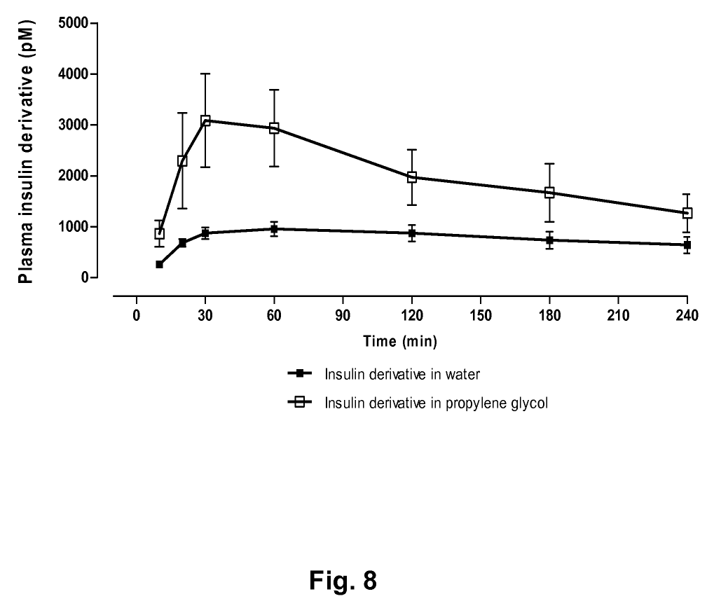

The insulin derivative B29K(N(eps)Octadecanedioyl-gGlu-OEG-OEG) A14E B25H desB30 human insulin (60 nmol/kg) was dissolved in water or in propylene glycol.
Plasma exposure (in pM) was measured after injection into mid-jejunum of fasted male SPRD rats (mean±SEM, n=6). The results are shown in FIG. 8.

### Example 11

### Plasma Exposure of Derivatized Insulin Peptide Formulated in SEDDS

The insulin derivative B29K(N(eps)Octadecanedioyl-gGlu-OEG-OEG) A14E B25H desB30 human insulin was formulated in different pharmaceutical compositions consisting of the insulin derivative and:

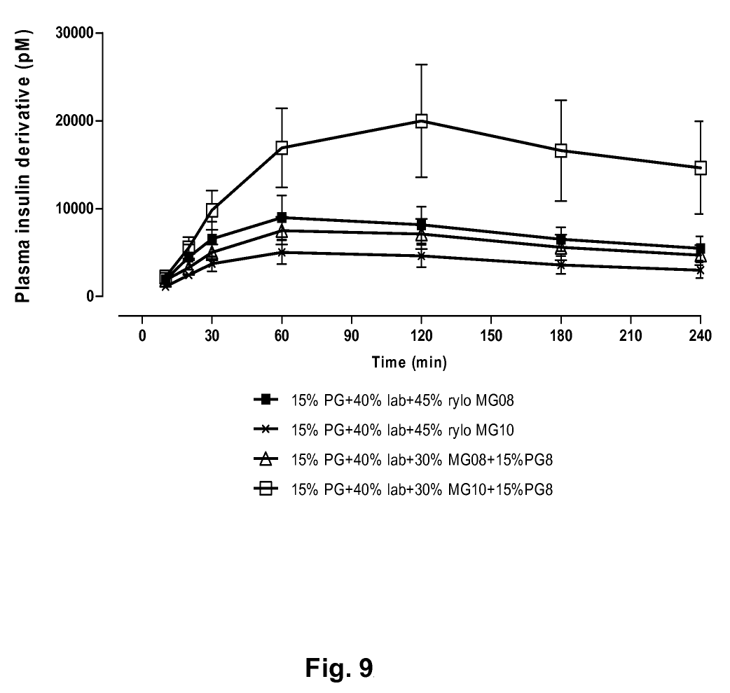

a) 15% propylene glycol and 40% Labrasol and 45% Rylo MG08 (glycerol caprylate), b) 15% propylene glycol, 40% Labrasol, 30% Rylo MG10 (glycerol caprate) and 15% propylene glycol caprylate, c) 15% propylene glycol, 40% Labrasol, 45% Rylo MG10 (glycerol caprate), and d) 15% propylene glycol, 40% Labrasol, 30% Rylo MG08 (glycerol caprylate), 15% propylene glycol caprylate.
The samples were prepared by the following method:
Lyophilized pH neutral powder of the insulin derivative B29K(N(eps)Octadecanedioyl-gGlu-OEG-OEG) A14E B25H desB30 human insulin was dissolved in propylene glycol at RT and after complete dissolution, the according lipid component and the according surfactant were added and mixed by magnetic stirring at RT for 5 to 10 minutes to result in clear homogenous liquids.
Plasma exposure (in pM) of the insulin derivative in the different pharmaceutical compositions was determined after intestinal injection of 60 nmol/kg (0.4 ml/kg) into the mid-jejunum of fasted male SPRD rats (mean±SEM, n=5-6). The results are shown in FIG. 9.

### Example 12

### High Insulin Derivative Drug Loads in Water-Free SEDDS Formulation

Various amounts of the insulin derivative B29K(N(eps)Octadecanedioyl-gGlu-OEG-OEG) A14E B25H desB30 human insulin were formulated in SEDDS:
1% (w/w) insulin derivative in SEDDS:
10 mg of insulin derivative were first dissolved in 150 mg of propylene glycol, and after dissolution mixed with 400 mg Labrasol and 440 mg Rylo MG08 at RT.
2% (w/w) insulin derivative in SEDDS:
20 mg of insulin derivative were first dissolved in 150 mg of propylene glycol, and after dissolution mixed with 400 mg Labrasol and 430 mg Rylo MG08 at RT.
3% (w/w) insulin derivative in SEDDS:
30 mg of insulin derivative were first dissolved in 150 mg of propylene glycol, and after dissolution mixed with 400 mg Labrasol and 420 mg Rylo MG08 at RT.
4% (w/w) insulin derivative in SEDDS:
40 mg of insulin derivative were first dissolved in 150 mg of propylene glycol, and after dissolution mixed with 400 mg Labrasol and 410 mg Rylo MG08 at RT.
9% (w/w) insulin derivative in SEDDS:
90 mg of insulin derivative were first dissolved in 150 mg of propylene glycol, and after dissolution mixed with 400 mg Labrasol and 360 mg Rylo MG08 at RT.

All SEDDS resulted in clear, homogenous solution like formulations with the insulin derivative completely dissolved in the formulation. Surprisingly high drug loads of the insulin derivative B29K(N(eps)Octadecanedioyl-gGlu-OEG-OEG) A14E B25H desB30 human insulin could be dissolved in the water-free pharmaceutical compositions comprising propylene glycol, Labrasol and glycerol mono caprylate (Rylo MG08 Pharma).

### Example 13

### Blood Glucose Lowering Effect after Administration of Insulin Derivative in SEDDS to Dogs

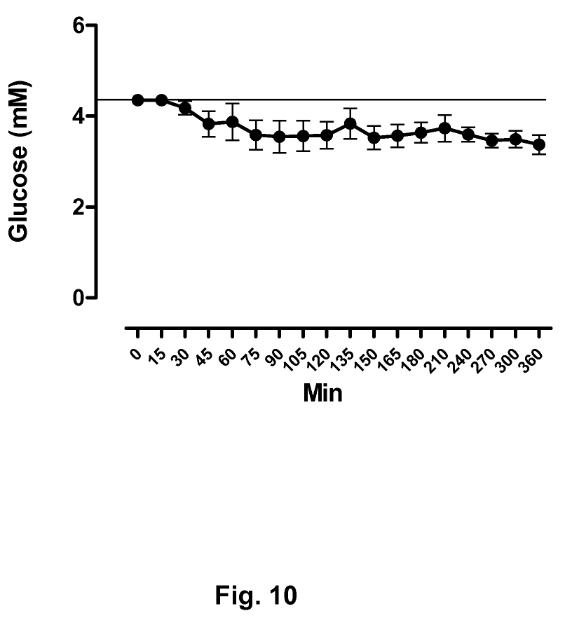

Blood glucose lowering effect in male beagle dogs (17 kg body weight) was measured after peroral administration of an enteric coated HPMC capsule containing 180 nmol/kg of the insulin derivative B29K(N(eps)Octadecanedioyl-gGlu-OEG-OEG) A14E B25H desB30 human insulin (60 nmol/kg) formulated with 15% propylene glycol, 40% Labrasol and 45% Capmul MCM (Glycerol caprylate/caprate).
The results are shown in FIG. 10.

### Example 14

### Plasma Exposure of Derivatized Insulin Peptide Formulated in SEDDS

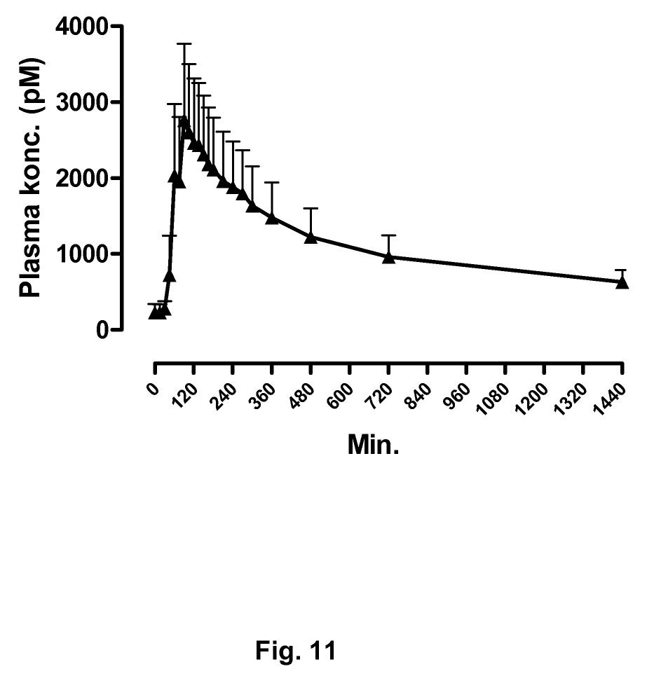

24 hour plasma exposure profile (in pM) of the insulin derivative B29K(N(eps)Octadecanedioyl-gGlu-OEG-OEG) A14E B25H desB30 human insulin in male beagle dogs (17 kg body weight) was measured after peroral administration of an enteric coated soft-gelatine capsule containing 30 nmol/kg of the insulin derivative dissolved in 15% propylene glycol, 40% Labrasol and 45% Rylo MG08 Pharma (Glycerol caprylate). The soft-gelatine capsules were coated with Eudragit L 30 D-55.
The results are shown in FIG. 11.

1. A water-free liquid pharmaceutical composition comprising a derivatized insulin peptide (a), at least one polar organic solvent (b) for the derivatized insulin peptide, at least one lipophilic component (c), and optionally at least one surfactant (d), wherein the pharmaceutical composition is in the form of a clear solution.

2. The pharmaceutical composition according to claim 1, which comprises at least one surfactant, wherein said pharmaceutical composition is spontaneously dispersible.

3. The pharmaceutical composition according to claim 1, which comprises less than 10% w/w water.

4. The pharmaceutical composition according to claim 1, wherein said polar organic solvent is selected from the group consisting of polyols.

5. The pharmaceutical composition according to claim 1, wherein the surfactant is a non ionic surfactant.

6. The pharmaceutical composition according to claim 1, wherein the lipophilic component is chosen such that a solution is obtained when the lipophilic component is mixed with propylene glycol.

7. The pharmaceutical composition according to claim 1, wherein the lipophilic component is a mono- and/or di-glyceride or propylene glycol caprylate.

8. The pharmaceutical composition according to claim 1, wherein the derivatized insulin peptide is an acylated insulin peptide.

9. The pharmaceutical composition according to claim 1, wherein the derivatized insulin peptide is a protease stabilised insulin which has an acyl moiety attached to the protease stabilised insulin, wherein the acyl moiety has the general formula:
Acy-AA1ₙ-AA2ₘ-AA3ₚ- (I),
wherein n is 0 or an integer in the range from 1 to 3;
m is 0 or an integer in the range from 1 to 10;
p is 0 or an integer in the range from 1 to 10;
Acy is a fatty acid or a fatty diacid comprising from about 8 to about 24 carbon atoms;
AA1 is a neutral linear or cyclic amino acid residue;
AA2 is an acidic amino acid residue;
AA3 is a neutral, alkyleneglycol-containing amino acid residue;
and wherein the order by which AA1, AA2 and AA3 appears in the formula can be interchanged independently.

10. The pharmaceutical composition according to claim 1, wherein the pharmaceutical composition is encapsulated in a hard or soft capsule.

11. The pharmaceutical composition according claim 10, wherein the hard or soft capsule is enteric coated.

12. (canceled)

13. A method of producing a pharmaceutical composition according to claim 1, said method comprising the steps of:
(a) dissolving the derivatized insulin peptide in the polar organic solvent and
(b) subsequently mixing with the lipophilic component and optionally with the surfactant and/or hydrophilic component.

14. (canceled)
